(19) United States
(12) Patent Application Publication
(10) Pub. No.: US 2010/0247482 A1
(43) Pub. Date: Sep. 30, 2010

(54) TIGHT JUNCTION MODULATOR PEPTIDES FOR ENHANCED MUCOSAL DELIVERY OF THERAPEUTIC COMPOUNDS

(75) Inventors: Kunyuan Cui, Bothell, WA (US); Shu-Chih Chen, Woodinville, WA (US); Michael E. Houston, JR., Sammamish, WA (US); Steven C. Quay, Woodinville, WA (US)

Correspondence Address: Eckman Basu LLP, 2225 E. Bayshore Road, Suite 200, Palo Alto, CA, 94303-3220, US

(73) Assignee: NASTECH PHARMACEUTICAL COMPANY INC., Bothell, WA (US)

(21) Appl. No.: 12/108,452

(22) Filed: Apr. 23, 2008

Related U.S. Application Data
(63) Continuation of application No. 11/233,239, filed on Sep. 21, 2005, now abandoned.
(60) Provisional application No. 60/612,121, filed on Sep. 21, 2004.
(60) Provisional application No. 60/667,835, filed on Apr. 1, 2005.
(60) Provisional application No. 60/703,291, filed on Jul. 27, 2005.

(51) Int. Cl. A61K 38/20; C07K 14/435; C07K 7/08; A61K 38/17; A61K 38/21; A61K 39/00; A61K 39/395; A61P 31/12; A61P 35/00; A61P 37/02; A61P 19/10; A61P 29/00; A61P 13/12; A61P 11/00; A61P 9/00; A61P 7/12; A61P 5/00; A61P 3/00; A61K 38/46; A61K 38/44; A61K 38/48

(52) U.S. Cl. 424/85.2; 530/324; 530/325; 530/326; 514/1.2; 424/85.6; 424/85.7; 424/184.1; 424/141.1; 424/133.1; 424/94.6; 424/130.1; 424/94.4; 424/94.64; 424/94.65

(57) ABSTRACT

Compositions and methods are provided that include a biologically active agent and a permeabilizing agent effective to enhance mucosal delivery of the biologically active agent in a mammalian subject, in which the permeabilizing peptide is a PN159 analog or conjugate.

This application is a continuation claiming the benefit under 35 U.S.C. §120 of copending U.S. patent application Ser. No. 11/233,239, filed Sep. 21, 2005, which claimed the benefit under 35 U.S.C. §119(e) of U.S. Provisional Application No. 60/612,121, filed Sep. 21, 2004, U.S. Provisional Application No. 60/667,835, filed Apr. 1, 2005, and U.S. Provisional Application No. 60/703,291, filed Jul. 27, 2005, the contents of each of the foregoing applications being incorporated herein by reference in its entirety.

### BACKGROUND OF THE INVENTION

A major disadvantage of drug administration by injection is that trained personnel are often required to administer the drug. For self-administered drugs, many patients are reluctant or unable to give themselves injections on a regular basis. Injection is also associated with increased risks of infection. Other disadvantages of drug injection include variability of delivery results between individuals, as well as unpredictable intensity and duration of drug action.

Despite these noted disadvantages, injection remains the only approved delivery mode for a large assemblage of important therapeutic compounds. These include conventional drugs, as well as a rapidly expanding list of peptide and protein biotherapeutics. Delivery of these compounds via alternate routes of administration, for example, oral, nasal and other mucosal routes, often yields variable results and adverse side effects, and fails to provide suitable bioavailabilty. For macromolecular species in particular, especially peptide and protein therapeutics, alternate routes of administration are limited by susceptibility to inactivation and poor absorption across mucosal barriers.

Mucosal administration of therapeutic compounds may offer certain advantages over injection and other modes of administration, for example in terms of convenience and speed of delivery, as well as by reducing or elimination compliance problems and side effects that attend delivery by injection. However, mucosal delivery of biologically active agents is limited by mucosal barrier functions and other factors. For these reasons, mucosal drug administration typically requires larger amounts of drug than administration by injection. Other therapeutic compounds, including large molecule drugs, peptides and proteins, are often refractory to mucosal delivery.

The ability of drugs to permeate mucosal surfaces, unassisted by delivery-enhancing agents, appears to be related to a number of factors, including molecular size, lipid solubility, and ionization. Small molecules, less than about 300-1,000 daltons, are often capable of penetrating mucosal barriers, however, as molecular size increases, permeability decreases rapidly. Lipid-soluble compounds are generally more permeable through mucosal surfaces than are non-lipid-soluble molecules. Peptides and proteins are poorly lipid soluble, and hence exhibit poor absorption characteristics across mucosal surfaces.

In addition to their poor intrinsic permeability, large macromolecular drugs, including proteins and peptides, are often subject to limited diffusion, as well as lumenal and cellular enzymatic degradation and rapid clearance at mucosal sites. These mucosal sites generally serve as a first line of host defense against pathogens and other adverse environmental agents that come into contact with the mucosal surface. Mucosal tissues provide a substantial barrier to the free diffusion of macromolecules, while enzymatic activities present in mucosal secretions can severely limit the bioavailability of therapeutic agents, particularly peptides and proteins. At certain mucosal sites, such as the nasal mucosa, the typical residence time of proteins and other macromolecular species delivered is limited, e.g., to about 15-30 minutes or less, due to rapid mucociliary clearance.

In summary, previous attempts to successfully deliver therapeutic compounds, including small molecule drugs and protein therapeutics, via mucosal routes have suffered from a number of important and confounding deficiencies. These deficiencies point to a long-standing unmet need in the art for pharmaceutical formulations and methods of administering therapeutic compounds that are stable and well tolerated and that provide enhanced mucosal delivery, including to targeted tissues and physiological compartments such as central nervous system. More specifically, there is a need in the art for safe and reliable methods and compositions for mucosal delivery of therapeutic compounds for treatment of diseases and other adverse conditions in mammalian subjects. A related need exists for methods and compositions that will provide efficient delivery of macromolecular drugs via one or more mucosal routes in therapeutic amounts, which are fast acting, easily administered and have limited adverse side effects such as mucosal irritation or tissue damage.

In relation to these needs, an especially challenging need persists in the art for methods and compositions to enhance mucosal delivery of biotherapeutic compounds that will overcome mucosal epithelial barrier mechanisms. Selective permeability of mucosal epithelia has heretofore presented major obstacles to mucosal delivery of therapeutic macromolecules, including biologically active peptides and proteins. Accordingly, there remains a substantial unmet need in the art for new methods and tools to facilitate mucosal delivery of biotherapeutic compounds. In particular, there is a compelling need in the art for new methods and formulations to facilitate mucosal delivery of biotherapeutic compounds that have heretofore proven refractory to delivery across mucosal barriers. Elucidation of permeation enhancers with low toxicity continues to be an important endeavor.

A peptide was previously produced, synthesized, and used to study the interaction of bioactive helical amphipathic peptides with lipids (Steiner V, et al., 1991. J. Chromatogr. 586: 43-50). This peptide, hereinafter “PN159”, was found to have cell lytic ability and taken up by cells (Ha~llbrink M, et al., 2001. Biochim. Biophys. Acta 1515: 101-09). Subsequently, modifications of the PN159 peptide were tested for their effect on cellular uptake (Scheller A, et al., 1999. J. Pept. Sci. 5: 185-94).

Peptides capable modulating the function of epithelial tight junctions have been previously described (Johnson PH and Quay SC (2000) Expert Opin Drug Deliv 2:281-98). PN159 was identified as a novel tight junction modulating (TJM) peptide that is capable of reducing transepithelial electrical resistance (TER) across a tissue barrier and increase paracellular transport of 3,000 Da MW dextran with low cytotoxicity and high retention of cell viability.

### SUMMARY OF THE INVENTION

One aspect of the invention is a pharmaceutical formulation comprising a biologically active agent and a mucosal delivery-enhancing effective amount of a permeabilizing peptide that reversibly enhances mucosal epithelial transport of a biologically active agent in a mammalian subject, in which the permeabilizing peptide is PN159, PN159 analogues, conjugates of PN159, conjugates of PN159 analogues, or complexes thereof. In preferred embodiments of the invention, the permeabilizing peptide is selected from the group consisting of:

| | |
|---|---|
| NH2-KLALKLALKALKAALKLA-amide | (SEQ ID NO: 1) |
| NH2-klalklalkalkaalkla-amide | (SEQ ID NO: 2) |
| NH2-alklaaklaklalklalk-amide | (SEQ ID NO: 5) |
| NH2-RLAWRLALRALRAALRLA-amide | (SEQ ID NO: 13) |
| NH2-KLAWKLALKALKAALKLA-amide | (SEQ ID NO: 14) |
| NH2-KLAWKLALKALKAAWKLA-amide | (SEQ ID NO: 15) |
| NH2-KLAWKLAWKALKAAWKLA-amide | (SEQ ID NO: 16) |
| NH2-KALKLKAALALLAKLKLA-amide | (SEQ ID NO: 20) |
| and | |
| NH2-KALAALLKKAAKLLAALK-amide. | (SEQ ID NO: 22) |

In another aspect of the invention, the permeabilizing peptide is conjugated to at least one water soluble chain. In a preferred embodiment the water soluble chain is a poly(alkylene oxide) chain. In a more preferred embodiment the poly(alkylene oxide) chain is a polyethylene glycol (PEG) chain, which may have a molecular size between about 0.2 and about 200 kiloDaltons (kDa).

In another aspect of the invention, the enhancer of permeation decreases electrical resistance across a mucosal tissue barrier. In a preferred embodiment, the decrease in electrical resistance is at least 80% of the electrical resistance prior to applying the enhancer of permeation. In a related embodiment, the enhancer(s) of permeation increases permeability of the molecule across a mucosal tissue barrier, preferably at least two fold. In another embodiment, the increased permeability is paracellular. In another embodiment, the increased permeability results from modification of tight junctions. In an alternate embodiment, the increased permeability is transcellular, or a combination of trans- and paracellular.

In another aspect of the invention the mucosal tissue layer is comprised of an epithelial cell layer. In a preferred embodiment, the epithelial cell is selected from the group consisting of tracheal, bronchial, alveolar, nasal, pulmonary, gastrointestinal, epidermal or buccal, most preferably nasal.

In another aspect of the invention the biologically active agent is a peptide or protein. In a related embodiment, the peptide or protein is comprised of between 2 and 1000 amino acids. In a preferred embodiment, the peptide or protein is comprised of between 2 and 50 amino acids. In another embodiment, the peptide or protein is cyclic. In another embodiment, the peptide or protein forms dimers or higher-order oligomers via physical or chemical bonding. In a preferred embodiment, the peptide or protein is selected from the group comprising GLP-1, PYY₃₋₃₆, PTH₁₋₃₄ and Exendin-4. In another embodiment, the biologically active agent is a protein, preferably selected from the group consisting of beta-interferon, alpha-interferon, insulin, erythropoietin, G-CSF, and GM-CSF, growth hormone, and analogues of any of these.

Another aspect of the invention is a method of administering a molecule to an animal comprising preparing any of the formulations above, and bringing such formulation in contact with a mucosal surface of such animal. In a preferred embodiment, the mucosal surface is intranasal.

Another aspect of the invention is a dosage form comprising any of the formulations above, in which the dosage form is liquid, preferably in the form of droplets. Alternatively, the dosage form may be solid, either, to be reconstituted in liquid prior to administration, to be administered as a powder, or in the form of a capsule, tablet or gel.

Another aspect of the invention is a molecule that reversibly enhances mucosal epithelial transport of a biological agent in a mammalian subject, comprising PN159, PN159 analogues, conjugates of PN159, conjugates of PN159 analogues, or complexes thereof. In a preferred embodiment, the permeabilizing peptide is selected from the group consisting of

| | |
|---|---|
| NH2-KLALKLALKALKAALKLA-amide | (SEQ ID NO: 1) |
| NH2-klalklalkalkaalkla-amide | (SEQ ID NO: 2) |
| NH2-alklaaklaklalklalk-amide | (SEQ ID NO: 5) |
| NH2-RLAWRLALRALRAALRLA-amide | (SEQ ID NO: 13) |
| NH2-KLAWKLALKALKAALKLA-amide | (SEQ ID NO: 14) |
| NH2-KLAWKLALKALKAAWKLA-amide | (SEQ ID NO: 15) |
| NH2-KLAWKLAWKALKAAWKLA-amide | (SEQ ID NO: 16) |
| NH2-KALKLKAALALLAKLKLA-amide | (SEQ ID NO: 20) |
| and | |
| NH2-KALAALLKKAAKLLAALK-amide. | (SEQ ID NO: 22) |

In another aspect of the invention, the permeabilizing peptide is covalently linked to a single poly(alkylene oxide) chain, prefereably a polyethylene glycol (PEG) chain, most preferably, a PEG that has a molecular size between about 0.2 and about 200 kiloDaltons (kDa).

### BRIEF DESCRIPTION OF THE DRAWINGS

FIG. 1 illustrates the effects of PN159 on permeation of PTH₁₋₃₄, using PN159 with additional enhancers (Me-β-CD, DDPC, EDTA).

FIG. 2 illustrates the effects of PN159 on permeation of PTH₁₋₃₄, using PN159 without additional enhancers.

FIG. 3 illustrates the effects of PN159 on in vivo permeation of peptide YY.

FIG. 4 illustrates the effects of PN159 on permeation of an MC-4 receptor agonist.

FIG. 5 shows the effects of 25-100 μM PN159 on 40 mg/ml Galantamine lactate in vitro permeation of an epithelial monolayer.

FIG. 6 shows the chemical stability of TJM peptide at (A) 5° C., (B) 25° C., and (C) 40° C. (D) Arrhenius plot for various pH conditions, including thoese in A, B and C. Data are presented for pH 4.0, pH 7.3 and pH 9.0 as filled diamonds, open squares, and filled triangles, respectively.

### DETAILED DESCRIPTION OF THE INVENTION

The instant invention satisfies the foregoing needs and fulfills additional objects and advantages by providing novel permeabilizing peptides and novel pharmaceutical compositions that include a biologically active agent and one or more of the novel permeabilizing peptides effective to enhance mucosal delivery of the biologically active agent in a mammalian subject. The permeabilizing peptides of the invention include PN159, having the sequence NH2-KLALKLALKALKAALKLA-amide (SEQ ID NO: 1), analogues of PN159 disclosed herein, combinations of these analogues, conjugates of PN159, conjugates of PN159 analogues, and complexes thereof.

The permeabilizing peptides and related compositions and methods of the invention reversibly enhance mucosal epithelial paracellular transport, typically by modulating epithelial junctional structure and/or physiology at a mucosal epithelial surface in the subject. This effect typically involves inhibition by the permeabilizing agent of homotypic or heterotypic binding between epithelial membrane adhesive proteins of neighboring epithelial cells.

Epithelial cells provide a crucial interface between the external environment and mucosal and submucosal tissues and extracellular compartments. One of the most important functions of mucosal epithelial cells is to determine and regulate mucosal permeability. In this context, epithelial cells create selective permeability barriers between different physiological compartments. Selective permeability is the result of regulated transport of molecules through the cytoplasm (the transcellular pathway) and the regulated permeability of the spaces between the cells (the paracellular pathway).

Intercellular junctions between epithelial cells are known to be involved in both the maintenance and regulation of the epithelial barrier function, and cell-cell adhesion. The tight junction (TJ) of epithelial and endothelial cells is a particularly important cell-cell junction that regulates permeability of the paracellular pathway, and also divides the cell surface into apical and basolateral compartments. Tight junctions form continuous circumferential intercellular contacts between epithelial cells and create a regulated barrier to the paracellular movement of water, solutes, and immune cells. They also provide a second type of barrier that contributes to cell polarity by limiting exchange of membrane lipids between the apical and basolateral membrane domains.

Tight junctions are thought to be directly involved in barrier and fence functions of epithelial cells by creating an intercellular seal to generate a primary barrier against the diffusion of solutes through the paracellular pathway, and by acting as a boundary between the apical and basolateral plasma membrane domains to create and maintain cell polarity, respectively. Tight junctions are also implicated in the transmigration of leukocytes to reach inflammatory sites. In response to chemoattractants, leukocytes emigrate from the blood by crossing the endothelium and, in the case of mucosal infections, cross the inflamed epithelium. Transmigration occurs primarily along the paracellular rout and appears to be regulated via opening and closing of tight junctions in a highly coordinated and reversible manner.

Numerous proteins have been identified in association with TJs, including both integral and peripheral plasma membrane proteins. Current understanding of the complex structure and interactive functions of these proteins remains limited. Among the many proteins associated with epithelial junctions, several categories of trans-epithelial membrane proteins have been identified that may function in the physiological regulation of epithelial junctions. These include a number of “junctional adhesion molecules” (JAMs) and other TJ-associated molecules designated as occludins, claudins, and zonulin.

JAMs, occludin, and claudin extend into the paracellular space, and these proteins in particular have been contemplated as candidates for creating an epithelial barrier between adjacent epithelial cells and regulatable channels through epithelial cell layers. In one model, occludin, claudin, and JAM have been proposed to interact as homophilic binding partners to create a regulated barrier to paracellular movement of water, solutes, and immune cells between epithelial cells.

A cDNA encoding murine junctional adhesion molecule-1 (JAM-1) has been cloned and corresponds to a predicted type I transmembrane protein (comprising a single transmembrane domain) with a molecular weight of approximately 32-kD [Williams, et al., Molecular Immunology 36: 1175-1188 (1999); Gupta, et al., IUBMB Life, 50: 51-56 (2000); Ozaki, et al., J. Immunol. 163: 553-557 (1999); Martin-Padura, et al., J. Cell Biol 142: 117-127 (1998)]. The extracellular segment of the molecule comprises two Ig-like domains described as an amino terminal “VH-type” and a carboxy-terminal “C2-type” carboxy-terminal β-sandwich fold [Bazzoni et al., Microcirculation 8:143-152 (2001)].

Another proposed trans-membrane adhesive protein involved in epithelial tight junction regulation is occludin. Occludin is an approximately 65-kD type II transmembrane protein composed of four transmembrane domains, two extracellular loops, and a large C-terminal cytosolic domain [Furuse et al., J. Cell Biol. 123:1777-1788 (1993); Furuse et al., J Cell Biol 127:1617-1626 (1994)]. This topology has been confirmed by antibody accessibility studies [Van Itallie, and Anderson, J. Cell. Sci. 110: 1113-1121 (1997)].

Other cytoplasmic proteins that have been localized to epithelial junctions include zonulin, symplekin, cingulin, and 7H6. Zonulins reportedly are cytoplasmic proteins that bind the cytoplasmic tail of occludin. Representing this family of proteins are “ZO-1, ZO-2, and ZO-3”. Zonulin is postulated to be a human protein analogue of the Vibrio cholerae derived zonula occludens toxin (ZOT).

Zonulin likely plays a role in tight junction regulation during developmental, physiological, and pathological processes—including tissue morphogenesis, movement of fluid, macromolecules and leukocytes between the intestinal lumen and the interstitium, and inflammatory/autoimmune disorders. See, e.g., Wang, et al., J. Cell Sci., 113:4435-40 (2000); Fasano, et al., Lancet 355:1518-9 (2000); Fasano, Ann. N.Y. Acad. Sci., 915: 214-222 (2000). Zonulin expression increased in intestinal tissues during the acute phase of coeliac disease, a clinical condition in which tight junctions are opened and permeability is increased. Zonulin induces tight junction disassembly and a subsequent increase in intestinal permeability in non-human primate intestinal epithelia in vitro.

Comparison of amino acids in the active V. cholerae ZOT fragment and human zonulin identified a putative receptor binding domain within the N-terminal region of the two proteins. The ZOT biologically active domain increases intestinal permeability by interacting with a mammalian cell receptor with subsequent activation of intracellular signaling leading to the disassembly of the intercellular tight junction. The ZOT biologically active domain has been localized toward the carboxyl terminus of the protein and coincides with the predicted cleavage product generated by V. cholerae. This domain shares a putative receptor-binding motif with zonulin, the ZOT mammalian analogue. Amino acid comparison between the ZOT active fragment and zonulin, combined with site-directed mutagenesis experiments, suggest an octapeptide receptor-binding domain toward the amino terminus of processed ZOT and the amino terminus of zonulin, Di Pierro, et al., J. Biol. Chem., 276: 19160-19165 (2001). ZO-1 reportedly binds actin, AF-6, ZO-associated kinase (ZAK), fodrin, and α-catenin.

Permeabilizing peptides for use within the invention include natural or synthetic, therapeutically or prophylactically active, peptides (comprised of two or more covalently linked amino acids), proteins, peptide or protein fragments, peptide or protein analogs, peptide or protein mimetics, and chemically modified derivatives or salts of active peptides or proteins. Thus, as used herein, the term “permeabilizing peptide” will often be intended to embrace all of these active species, i.e., peptides and proteins, peptide and protein fragments, peptide and protein analogs, peptide and protein mimetics, and chemically modified derivatives and salts of active peptides or proteins. Often, the permeabilizing peptides or proteins are muteins that are readily obtainable by partial substitution, addition, or deletion of amino acids within a naturally occurring or native (e.g., wild-type, naturally occurring mutant, or allelic variant) peptide or protein sequence. Additionally, biologically active fragments of native peptides or proteins are included. Such mutant derivatives and fragments substantially retain the desired biological activity of the native peptide or proteins. In the case of peptides or proteins having carbohydrate chains, biologically active variants marked by alterations in these carbohydrate species are also included within the invention.

The permeabilizing peptides, proteins, analogs and mimetics for use within the methods and compositions of the invention are often formulated in a pharmaceutical composition comprising a mucosal delivery-enhancing or permeabilizing effective amount of the permeabilizing peptide, protein, analog or mimetic that reversibly enhances mucosal epithelial paracellular transport by modulating epithelial junctional structure and/or physiology in a mammalian subject.

### Biologically Active Agents

The methods and compositions of the present invention are directed toward enhancing mucosal, e.g., intranasal, delivery of a broad spectrum of biologically active agents to achieve therapeutic, prophylactic or other desired physiological results in mammalian subjects. As used herein, the term “biologically active agent” encompasses any substance that produces a physiological response when mucosally administered to a mammalian subject according to the methods and compositions herein. Useful biologically active agents in this context include therapeutic or prophylactic agents applied in all major fields of clinical medicine, as well as nutrients, cofactors, enzymes (endogenous or foreign), antioxidants, and the like. Thus, the biologically active agent may be water-soluble or water-insoluble, and may include higher molecular weight proteins, peptides, carbohydrates, glycoproteins, lipids, and/or glycolipids, nucleosides, polynucleotides, and other active agents.

Useful pharmaceutical agents within the methods and compositions of the invention include drugs and macromolecular therapeutic or prophylactic agents embracing a wide spectrum of compounds, including small molecule drugs, peptides, proteins, and vaccine agents. Exemplary pharmaceutical agents for use within the invention are biologically active for treatment or prophylaxis of a selected disease or condition in the subject. Biological activity in this context can be determined as any significant (i.e., measurable, statistically significant) effect on a physiological parameter, marker, or clinical symptom associated with a subject disease or condition, as evaluated by an appropriate in vitro or in vivo assay system involving actual patients, cell cultures, sample assays, or acceptable animal models.

The methods and compositions of the invention provide unexpected advantages for treatment of diseases and other conditions in mammalian subjects, which advantages are mediated, for example, by providing enhanced speed, duration, fidelity or control of mucosal delivery of therapeutic and prophylactic compounds to reach selected physiological compartments in the subject (e.g., into or across the nasal mucosa, into the systemic circulation or central nervous system (CNS), or to any selected target organ, tissue, fluid or cellular or extracellular compartment within the subject).

In various exemplary embodiments, the methods and compositions of the invention may incorporate one or more biologically active agent(s) selected from:

opiods or opiod antagonists, such as morphine, hydromorphone, oxymorphone, lovorphanol, levallorphan, codeine, nalmefene, nalorphine, nalozone, naltrexone, buprenorphine, butorphanol, and nalbufine;

corticosterones, such as cortisone, hydrocortisone, fludrocortisone, prednisone, prednisolone, methylprednisolone, triamcinolone, dexamethoasone, betamethoasone, paramethosone, and fluocinolone;

other anti-inflammatories, such as colchicine, ibuprofen, indomethacin, and piroxicam; anti-viral agents such as acyclovir, ribavarin, trifluorothyridine, Ara-A (Arabinofuranosyladenine), acylguanosine, nordeoxyguanosine, azidothymidine, dideoxyadenosine, and dideoxycytidine; antiandrogens such as spironolactone;

androgens, such as testosterone;

estrogens, such as estradiol;

progestins;

muscle relaxants, such as papaverine;

vasodilators, such as nitroglycerin, vasoactive intestinal peptide and calcitonin related gene peptide;

antihistamines, such as cyproheptadine;

agents with histamine receptor site blocking activity, such as doxepin, imipramine, and cimetidine;

antitussives, such as dextromethorphan; neuroleptics such as clozaril; antiarrhythmics;

antiepileptics;

enzymes, such as superoxide dismutase and neuroenkephalinase;

anti-fungal agents, such as amphotericin B, griseofulvin, miconazole, ketoconazole, tioconazol, itraconazole, and fluconazole;

antibacterials, such as penicillins, cephalosporins, tetracyclines, aminoglucosides, erythromicin, gentamicins, polymyxin B;

anti-cancer agents, such as 5-fluorouracil, bleomycin, methotrexate, and hydroxyurea, dideoxyinosine, floxuridine, 6-mercaptopurine, doxorubicin, daunorubicin, I-darubicin, taxol and paclitaxel (optionally provided in a bimodal emulsion, e.g., as described in U.S. patent application Ser. No. 09/631,246, filed by Quay on Aug. 2, 2000);

antioxidants, such as tocopherols, retinoids, carotenoids, ubiquinones, metal chelators, and phytic acid;

antiarrhythmic agents, such as quinidine; and

antihypertensive agents such as prazosin, verapamil, nifedipine, and diltiazem;

analgesics such as acetaminophen and aspirin;

monoclonal and polyclonal antibodies, including humanized antibodies, and antibody fragments;

anti-sense oligonucleotides; and

RNA, DNA and viral vectors comprising genes encoding therapeutic peptides and proteins.

In addition to these exemplary classes and species of active agents, the methods and compositions of the invention embrace any physiologically active agent, as well as any combination of multiple active agents, described above or elsewhere herein or otherwise known in the art, that is individually or combinatorially effective within the methods and compositions of the invention for treatment or prevention of a selected disease or condition in a mammalian subject (see, Physicians' Desk Reference, published by Medical Economics Company, a division of Litton Industries, Inc).

Regardless of the class of compound employed, the biologically active agent for use within the invention will be present in the compositions and methods of the invention in an amount sufficient to provide the desired physiological effect with no significant, unacceptable toxicity or other adverse side effects to the subject. The appropriate dosage levels of all biologically active agents will be readily determined without undue experimentation by the skilled artisan. Because the methods and compositions of the invention provide for enhanced delivery of the biologically active agent(s), dosage levels significantly lower than conventional dosage levels may be used with success. In general, the active substance will be present in the composition in an amount of from about 0.01% to about 50%, often between about 0.1% to about 20%, and commonly between about 1.0% to 5% or 10% by weight of the total intranasal formulation depending upon the particular substance employed.

As used herein, the terms biolotically active “peptide” and “protein” include polypeptides of various sizes, and do not limit the invention to amino acid polymers of any particular size. Peptides from as small as a few amino acids in length, to proteins of any size, as well as peptide-peptide, protein-protein fusions and protein-peptide fusions, are encompassed by the present invention, so long as the protein or peptide is biologically active in the context of eliciting a specific physiological, immunological, therapeutic, or prophylactic effect or response.

The instant invention provides novel formulations and coordinate administration methods for enhanced mucosal delivery of biologically active peptides and proteins. Illustrative examples of therapeutic peptides and proteins for use within the invention include, but are not limited to: tissue plasminogen activator (TPA), epidermal growth factor (EGF), fibroblast growth factor (FGF-acidic or basic), platelet derived growth factor (PDGF), transforming growth factor (TGF-alpha or beta), vasoactive intestinal peptide, tumor necrosis factor (TGF), hypothalmic releasing factors, prolactin, thyroid stimulating hormone (TSH), adrenocorticotropic hormone (ACTH), parathyroid hormone (PTH), follicle stimulating hormone (FSF), luteinizing hormone releasing (LHRH), endorphins, glucagon, calcitonin, oxytocin, carbetocin, aldoetecone, enkaphalins, somatostin, somatotropin, somatomedin, gonadotrophin, estrogen, progesterone, testosterone, alpha-melanocyte stimulating hormone, non-naturally occurring opiods, lidocaine, ketoprofen, sufentainil, terbutaline, droperidol, scopolamine, gonadorelin, ciclopirox, olamine, buspirone, calcitonin, cromolyn sodium or midazolam, cyclosporin, lisinopril, captopril, delapril, cimetidine, ranitidine, famotidine, superoxide dismutase, asparaginase, arginase, arginine deaminease, adenosine deaminase ribonuclease, trypsin, chemotrypsin, and papain. Additional examples of useful peptides include, but are not limited to, bombesin, substance P, vasopressin, alpha-globulins, transferrin, fibrinogen, beta-lipoproteins, beta-globulins, prothrombin, ceruloplasmin, alpha₂-glycoproteins, alpha₂-globulins, fetuin, alpha₁-lipoproteins, alpha₁-globulins, albumin, prealbumin, and other bioactive proteins and recombinant protein products.

In more detailed aspects of the invention, methods and compositions are provided for enhanced mucosal delivery of specific, biologically active peptide or protein therapeutics to treat (i.e., to eliminate, or reduce the occurrence or severity of symptoms of) an existing disease or condition, or to prevent onset of a disease or condition in a subject identified to be at risk for the subject disease or condition. Biologically active peptides and proteins that are useful within these aspects of the invention include, but are not limited to hematopoietics; antiinfective agents; antidementia agents; antiviral agents; antitumoral agents; antipyretics; analgesics; antiinflammatory agents; antiulcer agents; antiallergic agents; antidepressants; psychotropic agents; cardiotonics; antiarrythmic agents; vasodilators; antihypertensive agents such as hypotensive diuretics; antidiabetic agents; anticoagulants; cholesterol lowering agents; therapeutic agents for osteoporosis; hormones; antibiotics; vaccines; and the like.

Biologically active peptides and proteins for use within these aspects of the invention include, but are not limited to, cytokines; peptide hormones; growth factors; factors acting on the cardiovascular system; cell adhesion factors; factors acting on the central and peripheral nervous systems; factors acting on humoral electrolytes and hemal organic substances; factors acting on bone and skeleton growth or physiology; factors acting on the gastrointestinal system; factors acting on the kidney and urinary organs; factors acting on the connective tissue and skin; factors acting on the sense organs;

factors acting on the immune system; factors acting on the respiratory system; factors acting on the genital organs; and various enzymes.

For example, hormones which may be administered within the methods and compositions of the present invention include androgens, estrogens, prostaglandins, somatotropins, gonadotropins, interleukins, steroids and cytokines

Vaccines which may be administered within the methods and compositions of the present invention include bacterial and viral vaccines, such as vaccines for hepatitis, influenza, respiratory syncytial virus (RSV), parainfluenza virus (PIV), tuberculosis, canary pox, chicken pox, measles, mumps, rubella, pneumonia, and human immunodeficiency virus (HIV).

Bacterial toxoids which may be administered within the methods and compositions of the present invention include diphtheria, tetanus, pseudonomas and mycobactrium tuberculosis.

Examples of specific cardiovascular or thromobolytic agents for use within the invention include hirugen, hirulos and hirudine.

Antibody reagents that are usefully administered with the present invention include monoclonal antibodies, polyclonal antibodies, humanized antibodies, antibody fragments, fusions and multimers, and immunoglobins.

As used herein, the term “conservative amino acid substitution” refers to the general interchangeability of amino acid residues having similar side chains. For example, a commonly interchangeable group of amino acids having aliphatic side chains is alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Examples of conservative substitutions include the substitution of a non-polar (hydrophobic) residue such as isoleucine, valine, leucine or methionine for another. Likewise, the present invention contemplates the substitution of a polar (hydrophilic) residue such as between arginine and lysine, between glutamine and asparagine, and between threonine and serine. Additionally, the substitution of a basic residue such as lysine, arginine or histidine for another or the substitution of an acidic residue such as aspartic acid or glutamic acid for another is also contemplated. Exemplary conservative amino acids substitution groups are: valine-leucine-isoleucine, phenylalanine-tyrosine, lysine-arginine, alanine-valine, and asparagine-glutamine.

The term biologically active peptide or protein analog further includes modified forms of a native peptide or protein incorporating stereoisomers (e.g., D-amino acids) of the twenty conventional amino acids, or unnatural amino acids such as α,α-disubstituted amino acids, N-alkyl amino acids, lactic acid. These and other unconventional amino acids may also be substituted or inserted within native peptides and proteins useful within the invention. Examples of unconventional amino acids include: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, ω-N-methylarginine, and other similar amino acids and imino acids (e.g., 4-hydroxyproline). In addition, biologically active peptide or protein analogs include single or multiple substitutions, deletions and/or additions of carbohydrate, lipid and/or proteinaceous moieties that occur naturally or artificially as structural components of the subject peptide or protein, or are bound to or otherwise associated with the peptide or protein.

In one aspect, peptides (including polypeptides) useful within the invention are modified to produce peptide mimetics by replacement of one or more naturally occurring side chains of the 20 genetically encoded amino acids (or D amino acids) with other side chains, for instance with groups such as alkyl, lower alkyl, cyclic 4-, 5-, 6-, to 7-membered alkyl, amide, amide lower alkyl, amide di(lower alkyl), lower alkoxy, hydroxy, carboxy and the lower ester derivatives thereof, and with 4-, 5-, 6-, to 7-membered heterocyclics. For example, proline analogs can be made in which the ring size of the proline residue is changed from 5 members to 4, 6, or 7 members. Cyclic groups can be saturated or unsaturated, and if unsaturated, can be aromatic or non-aromatic. Heterocyclic groups can contain one or more nitrogen, oxygen, and/or sulphur heteroatoms. Examples of such groups include the furazanyl, furyl, imidazolidinyl, imidazolyl, imidazolinyl, isothiazolyl, isoxazolyl, morpholinyl (e.g. morpholino), oxazolyl, piperazinyl (e.g. 1-piperazinyl), piperidyl (e.g. 1-piperidyl, piperidino), pyranyl, pyrazinyl, pyrazolidinyl, pyrazolinyl, pyrazolyl, pyridazinyl, pyridyl, pyrimidinyl, pyrrolidinyl (e.g. 1-pyrrolidinyl), pyrrolinyl, pyrrolyl, thiadiazolyl, thiazolyl, thienyl, thiomorpholinyl (e.g. thiomorpholino), and triazolyl. These heterocyclic groups can be substituted or unsubstituted. Where a group is substituted, the substituent can be alkyl, alkoxy, halogen, oxygen, or substituted or unsubstituted phenyl.

Peptides and proteins, as well as peptide and protein analogs and mimetics, can also be covalently bound to one or more of a variety of nonproteinaceous polymers, e.g., polyethylene glycol, polypropylene glycol, or polyoxyalkenes, in the manner set forth in U.S. Pat. No. 4,640,835; U.S. Pat. No. 4,496,689; U.S. Pat. No. 4,301,144; U.S. Pat. No. 4,670,417; U.S. Pat. No. 4,791,192; or U.S. Pat. No. 4,179,337.

Other peptide and protein analogs and mimetics within the invention include glycosylation variants, and covalent or aggregate conjugates with other chemical moieties. Covalent derivatives can be prepared by linkage of functionalities to groups which are found in amino acid side chains or at the N- or C-termini, by means which are well known in the art. These derivatives can include, without limitation, aliphatic esters or amides of the carboxyl terminus, or of residues containing carboxyl side chains, O-acyl derivatives of hydroxyl group-containing residues, and N-acyl derivatives of the amino terminal amino acid or amino-group containing residues, e.g., lysine or arginine. Acyl groups are selected from the group of alkyl-moieties including C3 to C18 normal alkyl, thereby forming alkanoyl aroyl species. Covalent attachment to carrier proteins, e.g., immunogenic moieties may also be employed.

In addition to these modifications, glycosylation alterations of biologically active peptides and proteins can be made, e.g., by modifying the glycosylation patterns of a peptide during its synthesis and processing, or in further processing steps. Particularly preferred means for accomplishing this are by exposing the peptide to glycosylating enzymes derived from cells that normally provide such processing, e.g., mammalian glycosylation enzymes. Deglycosylation enzymes can also be successfully employed to yield useful modified peptides and proteins within the invention. Also embraced are versions of a native primary amino acid sequence which have other minor modifications, including phosphorylated amino acid residues, e.g., phosphotyrosine, phosphoserine, or phosphothreonine, or other moieties, including ribosyl groups or cross-linking reagents.

Peptidomimetics may also have amino acid residues that have been chemically modified by phosphorylation, sulfonation, biotinylation, or the addition or removal of other moieties, particularly those that have molecular shapes similar to phosphate groups.

One can cyclize active peptides for use within the invention, or incorporate a desamino or descarboxy residue at the termini of the peptide, so that there is no terminal amino or carboxyl group, to decrease susceptibility to proteases, or to restrict the conformation of the peptide. C-terminal functional groups among peptide analogs and mimetics of the present invention include amide, amide lower alkyl, amide di(lower alkyl), lower alkoxy, hydroxy, and carboxy, and the lower ester derivatives thereof, and the pharmaceutically acceptable salts thereof.

A variety of additives, diluents, bases and delivery vehicles are provided within the invention that effectively control water content to enhance protein stability. These reagents and carrier materials effective as anti-aggregation agents in this sense include, for example, polymers of various functionalities, such as polyethylene glycol, dextran, diethylaminoethyl dextran, and carboxymethyl cellulose, which significantly increase the stability and reduce the solid-phase aggregation of peptides and proteins admixed therewith or linked thereto. In some instances, the activity or physical stability of proteins can also be enhanced by various additives to aqueous solutions of the peptide or protein drugs. For example, additives, such as polyols (including sugars), amino acids, proteins such as collagen and gelatin, and various salts may be used.

Certain additives, in particular sugars and other polyols, also impart significant physical stability to dry, e.g., lyophilized proteins. These additives can also be used within the invention to protect the proteins against aggregation not only during lyophilization but also during storage in the dry state. For example sucrose and Ficoll 70 (a polymer with sucrose units) exhibit significant protection against peptide or protein aggregation during solid-phase incubation under various conditions. These additives may also enhance the stability of solid proteins embedded within polymer matrices.

Yet additional additives, for example sucrose, stabilize proteins against solid-state aggregation in humid atmospheres at elevated temperatures, as may occur in certain sustained-release formulations of the invention. Proteins such as gelatin and collagen also serve as stabilizing or bulking agents to reduce denaturation and aggregation of unstable proteins in this context. These additives can be incorporated into polymeric melt processes and compositions within the invention. For example, polypeptide microparticles can be prepared by simply lyophilizing or spray drying a solution containing various stabilizing additives described above. Sustained release of unaggregated peptides and proteins can thereby be obtained over an extended period of time.

Various additional preparative components and methods, as well as specific formulation additives, are provided herein which yield formulations for mucosal delivery of aggregation-prone peptides and proteins, wherein the peptide or protein is stabilized in a substantially pure, unaggregated form. A range of components and additives are contemplated for use within these methods and formulations. Exemplary of these anti-aggregation agents are linked dimers of cyclodextrins (CDs), which selectively bind hydrophobic side chains of polypeptides. These CD dimers have been found to bind to hydrophobic patches of proteins in a manner that significantly inhibits aggregation. This inhibition is selective with respect to both the CD dimer and the protein involved. Such selective inhibition of protein aggregation provides additional advantages within the intranasal delivery methods and compositions of the invention. Additional agents for use in this context include CD trimers and tetramers with varying geometries controlled by the linkers that specifically block aggregation of peptides and proteins [Breslow et al., J. Am. Chem. Soc. 118:11678-11681 (1996); Breslow et al., PNAS USA 94:11156-11158 (1997)].

### Charge Modifying and PH Control Agents and Methods

To improve the transport characteristics of biologically active agents (e.g., macromolecular drugs, peptides or proteins) for enhanced delivery across hydrophobic mucosal membrane barriers, the invention also provides techniques and reagents for charge modification of selected biologically active agents or delivery-enhancing agents described herein. In this regard, the relative permeabilities of macromolecules is generally be related to their partition coefficients. The degree of ionization of molecules, which is dependent on the pKₐ, of the molecule and the pH at the mucosal membrane surface, also affects permeability of the molecules. Permeation and partitioning of biologically active agents and permeabilizing agents for mucosal delivery may be facilitated by charge alteration or charge spreading of the active agent or permeabilizing agent, which is achieved, for example, by alteration of charged functional groups, by modifying the pH of the delivery vehicle or solution in which the active agent is delivered, or by coordinate administration of a charge- or pH-altering reagent with the active agent.

### Preservatives

Preservative such as chlorobutanol, methyl paraben, propyl paraben, sodium benzoate (0.5%), phenol, cresol, p-chloro-m-cresol, phenylethyl alcohol, benzyl alcohol, phenylmercuric acetate, phenylmercuric borate, phenylmercuric nitrate, thimerosal, sorbic acid, benzethonium chloride or benzylkonium chloride can be added to the formulations of the invention to inhibit microbial growth.

### pH and Buffering Systems

The pH is generally regulated using a buffer such as a system comprised of citric acid and a citrate salt(s), such as sodium citrate. Additional suitable buffer systems include acetic acid and an acetate salt system, succinic acid and a succinate salt system, malic acid and a malic salt system, and gluconic acid and a gluconate salt system. Alternatively, buffer systems comprised of mixed acid/salt systems can be employed, such as an acetic acid and sodium citrate system, a citrate acid, sodium acetate system, and a citric acid, sodium citrate, sodium benzoate system. For any buffer system, additional acids, such as hydrochloric acid, and additional bases, such as sodium hydroxide, may be added for final pH adjustment.

Additional Agents for Modulating Epithelial Junction Structure and/or Physiology

Epithelial tight junctions are generally impermeable to molecules with radii of approximately 15 angstroms, unless treated with junctional physiological control agents that stimulate substantial junctional opening as provided within the instant invention. Among the “secondary” tight junctional regulatory components that will serve as useful targets for secondary physiological modulation within the methods and compositions of the invention, the ZO1-ZO2 heterodimeric complex has shown itself amenable to physiological regulation by exogenous agents that can readily and effectively alter paracellular permeability in mucosal epithelia. On such agent that has been extensively studied is the bacterial toxin from Vibrio cholerae known as the “zonula occludens toxin” (ZOT). See, also WO 96/37196; U.S. Pat. Nos. 5,945,510; 5,948,629; 5,912,323; 5,864,014; 5,827,534; 5,665,389; and 5,908,825. Thus, ZOT and other agents that modulate the ZO1-ZO2 complex will be combinatorially formulated or coordinately administered with one or more biologically active agents.

### Formulation and Administration

Mucosal delivery formulations of the present invention comprise the biologically active agent to be administered typically combined together with one or more pharmaceutically acceptable carriers and, optionally, other therapeutic ingredients. The carrier(s) must be “pharmaceutically acceptable” in the sense of being compatible with the other ingredients of the formulation and not eliciting an unacceptable deleterious effect in the subject. Such carriers are described herein above or are otherwise well known to those skilled in the art of pharmacology. Desirably, the formulation should not include substances such as enzymes or oxidizing agents with which the biologically active agent to be administered is known to be incompatible. The formulations may be prepared by any of the methods well known in the art of pharmacy.

The compositions and methods of the invention may be administered to subjects by a variety of mucosal administration modes, including by oral, rectal, vaginal, intranasal, intrapulmonary, or transdermal delivery, or by topical delivery to the eyes, ears, skin or other mucosal surfaces. Compositions according to the present invention are often administered in an aqueous solution as a nasal or pulmonary spray and may be dispensed in spray form by a variety of methods known to those skilled in the art. Preferred systems for dispensing liquids as a nasal spray are disclosed in U.S. Pat. No. 4,511,069. Such formulations may be conveniently prepared by dissolving compositions according to the present invention in water to produce an aqueous solution, and rendering said solution sterile. The formulations may be presented in multi-dose containers, for example in the sealed dispensing system disclosed in U.S. Pat. No. 4,511,069. Other suitable nasal spray delivery systems have been described in Transdermal Systemic Medication, Y. W. Chien Ed., Elsevier Publishers, New York, 1985; and in U.S. Pat. No. 4,778,810. Additional aerosol delivery forms may include, e.g., compressed air-, jet-, ultrasonic-, and piezoelectric nebulizers, which deliver the biologically active agent dissolved or suspended in a pharmaceutical solvent, e.g., water, ethanol, or a mixture thereof.

To formulate compositions for mucosal delivery within the present invention, the biologically active agent can be combined with various pharmaceutically acceptable additives, as well as a base or carrier for dispersion of the active agent(s). Desired additives include, but are not limited to, pH control agents, such as arginine, sodium hydroxide, glycine, hydrochloric acid, citric acid, etc. In addition, local anesthetics (e.g., benzyl alcohol), isotonizing agents (e.g., sodium chloride, mannitol, sorbitol), adsorption inhibitors (e.g., Tween 80), solubility enhancing agents (e.g., cyclodextrins and derivatives thereof), stabilizers (e.g., serum albumin), and reducing agents (e.g., glutathione) can be included. When the composition for mucosal delivery is a liquid, the tonicity of the formulation, as measured with reference to the tonicity of 0.9% (w/v) physiological saline solution taken as unity, is typically adjusted to a value at which no substantial, irreversible tissue damage will be induced in the nasal mucosa at the site of administration. Generally, the tonicity of the solution is adjusted to a value of about 1/3 to 3, more typically 1/2 to 2, and most often 3/4 to 1.7.

The biologically active agent may be dispersed in a base or vehicle, which may comprise a hydrophilic compound having a capacity to disperse the active agent and any desired additives. The base may be selected from a wide range of suitable carriers, including but not limited to, copolymers of polycarboxylic acids or salts thereof, carboxylic anhydrides (e.g. maleic anhydride) with other monomers (e.g. methyl (meth)acrylate, acrylic acid, etc.), hydrophilic vinyl polymers such as polyvinyl acetate, polyvinyl alcohol, polyvinylpyrrolidone, cellulose derivatives such as hydroxymethylcellulose, hydroxypropylcellulose, etc., and natural polymers such as chitosan, collagen, sodium alginate, gelatin, hyaluronic acid, and nontoxic metal salts thereof. Often, a biodegradable polymer is selected as a base or carrier, for example, polylactic acid, poly(lactic acid-glycolic acid) copolymer, polyhydroxybutyric acid, poly(hydroxybutyric acid-glycolic acid) copolymer and mixtures thereof. Alternatively or additionally, synthetic fatty acid esters such as polyglycerin fatty acid esters, sucrose fatty acid esters, etc. can be employed as carriers. Hydrophilic polymers and other carriers can be used alone or in combination, and enhanced structural integrity can be imparted to the carrier by partial crystallization, ionic bonding, crosslinking and the like. The carrier can be provided in a variety of forms, including, fluid or viscous solutions, gels, pastes, powders, microspheres and films for direct application to the nasal mucosa. The use of a selected carrier in this context may result in promotion of absorption of the biologically active agent.

The biologically active agent can be combined with the base or carrier according to a variety of methods, and release of the active agent may be by diffusion, disintegration of the carrier, or associated formulation of water channels. In some circumstances, the active agent is dispersed in microcapsules (microspheres) or nanocapsules (nanospheres) prepared from a suitable polymer, e.g., isobutyl 2-cyanoacrylate (see, e.g., Michael et al., J. Pharmacy Pharmacol. 43: 1-5, 1991), and dispersed in a biocompatible dispersing medium applied to the nasal mucosa, which yields sustained delivery and biological activity over a protracted time.

To further enhance mucosal delivery of pharmaceutical agents within the invention, formulations comprising the active agent may also contain a hydrophilic low molecular weight compound as a base or excipient. Such hydrophilic low molecular weight compounds provide a passage medium through which a water-soluble active agent, such as a physiologically active peptide or protein, may diffuse through the base to the body surface where the active agent is absorbed. The hydrophilic low molecular weight compound optionally absorbs moisture from the mucosa or the administration atmosphere and dissolves the water-soluble active peptide. The molecular weight of the hydrophilic low molecular weight compound is generally not more than 10000 and preferably not more than 3000. Exemplary hydrophilic low molecular weight compound include polyol compounds, such as oligo-, di- and monosaccarides such as sucrose, mannitol, lactose, L-arabinose, D-erythrose, D-ribose, D-xylose, D-mannose, D-galactose, lactulose, cellobiose, gentibiose, glycerin and polyethylene glycol. Other examples of hydrophilic low molecular weight compounds useful as carriers within the invention include N-methylpyrrolidone, and alcohols (e.g. oligovinyl alcohol, ethanol, ethylene glycol, propylene glycol, etc.) These hydrophilic low molecular weight compounds can be used alone or in combination with one another or with other active or inactive components of the intranasal formulation.

The compositions of the invention may alternatively contain as pharmaceutically acceptable carriers substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc. For solid compositions, conventional nontoxic pharmaceutically acceptable carriers can be used which include, for example, pharmaceutical grades of mannitol, lactose, starch, magnesium stearate, sodium saccharin, talcum, cellulose, glucose, sucrose, magnesium carbonate, and the like.

In certain embodiments of the invention, the biologically active agent is administered in a time release formulation, for example in a composition which includes a slow release polymer. The active agent can be prepared with carriers that will protect against rapid release, for example a controlled release vehicle such as a polymer, microencapsulated delivery system or bioadhesive gel. Prolonged delivery of the active agent, in various compositions of the invention can be brought about by including in the composition agents that delay absorption, for example, aluminum monosterate hydrogels and gelatin.

The term “subject” as used herein means any mammalian patient to which the compositions of the invention may be administered.

### Kits

The instant invention also includes kits, packages and multicontainer units containing the above described pharmaceutical compositions, active ingredients, and/or means for administering the same for use in the prevention and treatment of diseases and other conditions in mammalian subjects. Briefly, these kits include a container or formulation that contains one or more biologically active agent formulated in a pharmaceutical preparation for mucosal delivery. The biologically active agent(s) is/are optionally contained in a bulk dispensing container or unit or multi-unit dosage form. Optional dispensing means may be provided, for example a pulmonary or intranasal spray applicator. Packaging materials optionally include a label or instruction indicating that the pharmaceutical agent packaged therewith can be used mucosally, e.g., intranasally, for treating or preventing a specific disease or condition.

The above disclosure generally describes the present invention, which is further exemplified by the following examples. These examples are described solely for purposes of illustration, and are not intended to limit the scope of the invention. Although specific terms and values have been employed herein, such terms and values will likewise be understood as exemplary and non-limiting to the scope of the invention.

### Example 1

### Mucosal Delivery

### Permeation Kinetics and Cytotoxicity

Organotypic Model

The following methods are generally useful for evaluating mucosal delivery parameters, kinetics and side effects for a biologically active therapeutic agent and a mucosal delivery-enhancing effective amount of a permeabilizing peptide that reversibly enhances mucosal epithelial paracellular transport by modulating epithelial junctional structure and/or physiology in a mammalian subject.

The EpiAirway™ system was developed by MatTek Corp (Ashland, Mass.) as a model of the pseudostratified epithelium lining the respiratory tract. The epithelial cells are grown on porous membrane-bottomed cell culture inserts at an air-liquid interface, which results in differentiation of the cells to a highly polarized morphology. The apical surface is ciliated with a microvillous ultrastructure and the epithelium produces mucus (the presence of mucin has been confirmed by immunoblotting). The inserts have a diameter of 0.875 cm, providing a surface area of 0.6 cm². The cells are plated onto the inserts at the factory approximately three weeks before shipping. One “kit” consists of 24 units.

A. On arrival, the units are placed onto sterile supports in 6-well microplates. Each well receives 5 mL of proprietary culture medium. This DMEM-based medium is serum free but is supplemented with epidermal growth factor and other factors. The medium is always tested for endogenous levels of any cytokine or growth factor which is being considered for intranasal delivery, but has been free of all cytokines and factors studied to date except insulin. The 5 mL volume is just sufficient to provide contact to the bottoms of the units on their stands, but the apical surface of the epithelium is allowed to remain in direct contact with air. Sterile tweezers are used in this step and in all subsequent steps involving transfer of units to liquid-containing wells to ensure that no air is trapped between the bottoms of the units and the medium.

B. The units in their plates are maintained at 37° C. in an incubator in an atmosphere of 5% CO₂ in air for 24 hours. At the end of this time the medium is replaced with fresh medium and the units are returned to the incubator for another 24 hours.

Experimental Protocol—Permeation Kinetics

A. A “kit” of 24 EpiAirway™ units can routinely be employed for evaluating five different formulations, each of which is applied to quadruplicate wells. Each well is employed for determination of permeation kinetics (4 time points), transepithelial electrical resistance (TER). An additional set of wells is employed as controls, which are sham treated during determination of permeation kinetics, but are otherwise handled identically to the test sample-containing units for determinations of transepithelial resistance and viability.

B. In all experiments, the mucosal delivery formulation to be studied is applied to the apical surface of each unit in a volume of 100 μL, which is sufficient to cover the entire apical surface. An appropriate volume of the test formulation at the concentration applied to the apical surface (no more than 100 μL is generally needed) is set aside for subsequent determination of concentration of the active material by ELISA or other designated assay.

C. The units are placed in 6 well plates without stands for the experiment: each well contains 0.9 mL of medium which is sufficient to contact the porous membrane bottom of the unit but does not generate any significant upward hydrostatic pressure on the unit.

D. In order to minimize potential sources of error and avoid any formation of concentration gradients, the units are transferred from one 0.9 mL-containing well to another at each time point in the study. These transfers are made at the following time points, based on a zero time at which the 100 μL volume of test material was applied to the apical surface: 15 minutes, 30 minutes, 60 minutes, and 120 minutes.

E. In between time points the units in their plates are kept in the 37° C. incubator. Plates containing 0.9 mL medium per well are also maintained in the incubator so that minimal change in temperature occurs during the brief periods when the plates are removed and the units are transferred from one well to another using sterile forceps.

F. At the completion of each time point, the medium is removed from the well from which each unit was transferred, and aliquotted into two tubes (one tube receives 700 μL and the other 200 μL) for determination of the concentration of permeated test material and, in the event that the test material is cytotoxic, for release of the cytosolic enzyme, lactate dehydrogenase, from the epithelium. These samples are kept in the refrigerator if the assays are to be conducted within 24 hours, or the samples are subaliquotted and kept frozen at −80° C. until thawed once for assays. Repeated freeze-thaw cycles are to be avoided.

G. In order to minimize errors, all tubes, plates, and wells are prelabeled before initiating an experiment.

H. At the end of the 120 minute time point, the units are transferred from the last of the 0.9 mL containing wells to 24-well microplates, containing 0.3 mL medium per well. This volume is again sufficient to contact the bottoms of the units, but not to exert upward hydrostatic pressure on the units. The units are returned to the incubator prior to measurement of transepithelial resistance.

Experimental Protocol—Transepithelial Electrical Resistance

A. Respiratory airway epithelial cells form tight junctions in vivo as well as in vitro, and thereby restrict the flow of solutes across the tissue. These junctions confer a transepithelial resistance of several hundred ohms×cm² in excised airway tissues. In the MatTek EpiAirway™ units, the transepithelial electrical resistance (TER) is reported by the manufacturer to be routinely around 1000 ohms×cm². Data determined herein indicates that the TER of control EpiAirway™ units which have been sham-exposed during the sequence of steps in the permeation study is somewhat lower (700-800 ohms×cm²), but, since permeation of small molecules is proportional to the inverse of the TER, this value is still sufficiently high to provide a substantial barrier to permeation. The porous membrane-bottomed units without cells, conversely, provide only minimal transmembrane resistance (approximately 5-20 ohms×cm²).

B. Accurate determinations of TER require that the electrodes of the ohmmeter be positioned over a significant surface area above and below the membrane, and that the distance of the electrodes from the membrane be reproducibly controlled. The method for TER determination recommended by MatTek and employed for all experiments herein employs an “EVOM”™ epithelial voltohmmeter and an “ENDOHM”™ tissue resistance measurement chamber from World Precision Instruments, Inc., Sarasota, Fla.

C. The chamber is initially filled with Dulbecco's phosphate buffered saline (PBS) for at least 20 minutes prior to TER determinations in order to equilibrate the electrodes.

D. Determinations of TER are made with 1.5 mL of PBS in the chamber and 350 μL of PBS in the membrane-bottomed unit being measured. The top electrode is adjusted to a position just above the membrane of a unit containing no cells (but containing 350 μL of PBS) and then fixed to ensure reproducible positioning. The resistance of a cell-free unit is typically 5-20 ohms×cm² (“background resistance”).

E. Once the chamber is prepared and the background resistance is recorded, units in a 24-well plate that had just been employed in permeation determinations are removed from the incubator and individually placed in the chamber for TER determinations.

F. Each unit is first transferred to a petri dish containing PBS to ensure that the membrane bottom is moistened. An aliquot of 350 μL PBS is added to the unit and then carefully aspirated into a labeled tube to rinse the apical surface. A second wash of 350 μL PBS is then applied to the unit and aspirated into the same collection tube.

G. The unit is gently blotted free of excess PBS on its exterior surface only before being placed into the chamber (containing a fresh 1.5 mL aliquot of PBS). An aliquot of 350 μL PBS is added to the unit before the top electrode is placed on the chamber and the TER is read on the EVOM meter.

H. After the TER of the unit is read in the ENDOHM chamber, the unit is removed, the PBS is aspirated and saved, and the unit is returned with an air interface on the apical surface to a 24-well plate containing 0.3 mL medium per well.

I. The units are read in the following sequence: all sham-treated controls, followed by all formulation-treated samples, followed by a second TER reading of each of the sham-treated controls. All TER values are reported as a function of the surface area of the tissue.

TER was calculated as:

TER=(R_{I}−R_{b})×A

Where R_{I} is resistance of the insert with a membrane, R_{b} is the resistance of the blank insert, and A is the area of the membrane (0.6 cm²). The effect of pharmaceutical formulations comprising intranasal delivery-enhancing agents, for example, permeabilizing peptides as measured by TER across the EpiAirway™ Cell Membrane (mucosal epithelial cell layer). Permeabilizing peptides are applied to the EpiAirway™ Cell Membrane at a concentration of 1.0 mM. A decrease in TER value relative to the control value (control=approximately 1000 ohms-cm²; normalized to 100.) indicates a decrease in cell membrane resistance and an increase in mucosal epithelial cell permeability.

Experimental Protocol—LDH Assay

The amount of cell death was assayed by measuring the loss of lactate dehydrogenase (LDH) from the cells using a CytoTox 96 Cytoxicity Assay Kit (Promega Corp., Madison, Wis.). Fifty microliters of sample was loaded into a 96-well assay plates. Fresh, cell-free culture medium was used as a blank. 50 μl of substrate solution was added to each well and the plates incubated for 30 minutes at room temperature in the dark. Following incubation, 50 μl of stop solution was added to each well and the plates read on an optical density plate reader at 490 nm.

Experimental Protocol—EIA Method

EIA kit (p/n S-1178(EIAH6101) was purchased from Peninsula Laboratories Inc. (Division of BACHEM, San Carlos, Calif., 800-922-1516). 17×120 mm polypropylene conical tubes (p/n 352097, Falcon, Franklin Lakes, N.J.) were used for all sample preparations. Eight standards were used for PTH quantitation. The rest of the assay procedure was the same as the kit inserts.

### Example 2

### Epithelial Permeation Enhancement by PN159

The examples herein below demonstrate that permeation enhancing peptides of the invention, exemplified by PN159, enhance mucosal permeation to peptide therapeutic drugs, including PTH and Peptide YY. This permeation enhancing activity of the peptides of the invention, as evinced for PN159, can be equivalent to, or greater than, epithelial permeation enhancement achieved through the use of one or multiple small molecule permeation enhancers.

Peptide YY₃₋₃₆ (PYY₃₋₃₆) is a 34 amino acid peptide which has been the subject of numerous clinical trials. Mucosal delivery of this biologically active peptide can be enhanced in formulations that include small molecule permeation enhancers. Accordingly, the instant studies assessed whether the permeation enhancing peptides of the invention, exemplified by PN159, could replace the role of small molecule permeation enhancers to facilitate mucosal delivery of peptide YY. These studies included evaluation of in vitro effects of PN159 to decrease Transepithelial Electrical Resistance (TEER) and increase permeation of marker substances, as well as related in vivo studies that proved consistent with the in vitro results.

In the current example, the combination of PN159 with PTH is described. PTH can be the full length peptide (1-84), or a fragment such as (1-34). The formulation can also be a combination of PTH, a permeabilizing peptide, and one or more other permeation enhancers. The formulation may also contain buffers, tonicifying agents, pH adjustment agents, and peptide/protein stabilizers such as amino acids, sugars or polyols, polymers, and salts.

The instant study was designed to evaluate the effect of PN159 itself or in combination with additional permeation enhancers on PTH permeation. The PN159 concentrations evaluated are 25, 50, and 100 μM. The additional permeation enhancers are 45 mg/ml M-(3-CD, 1 mg/ml DDPC, and 1 mg/ml EDTA. Sorbitol was used as a tonicifier (146-190 mM) to adjust the osmolarity of formulations to 220 mOsm/kg. The formulation pH was fixed at 4.5. PTH was chosen as a model peptide in this example. 2 mg/ml PTH was combined with PN159 with or without additional permeation enhancers. The combination was tested using an in vitro epithelial tissue model to monitor PTH permeation, transepithelial electrical resistance (TER), and the cytotoxicity of the formulation by LDH assay.

Transepithelial Electrical Resistance

The results of TER measurements from the present studies show more than 80% TER reduction caused by PN159. Higher TER reduction was observed with increasing PN159 concentration. Media applied to the apical side did not reduce TER whereas triton X treated group showed significant TER reduction as expected.

Cytotoxicity

The data for LDH from the present studies shown no significant cytotoxicity was observed when cells were treated with 25-100 μM of PN159. Media applied to the apical side did not show cytotoxicity whereas the Triton X treated group showed significant cytotoxicity as expected.

Permeation

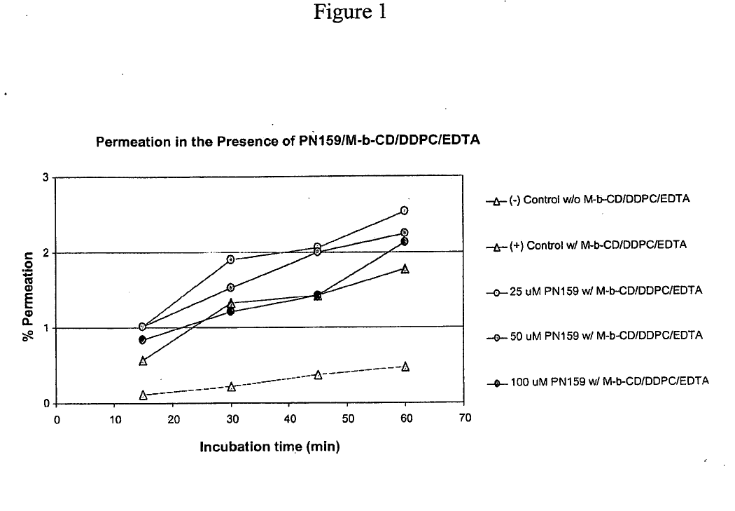

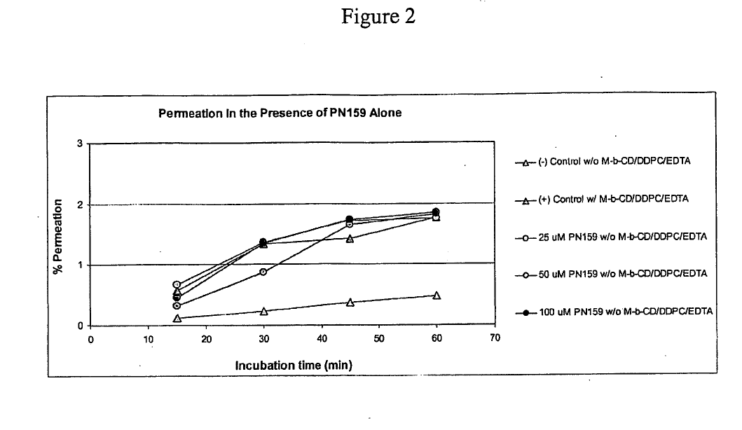

The PTH₁₋₃₄ permeation data for PN159 with and without additional enhancers are shown in FIGS. 1 and 2, respectively. Significant increase in PTH permeation was observed in the presence of PN159. No significant difference in % permeation was observed between 25, 50, and 100 μM PN159. Effect of PN159 on PTH permeation is comparable to 45/1/1 mg/ml M-β-CD/DDPC/EDTA. Additional increase in PTH permeation was observed with the combination of 45/1/1 mg/ml M-b-CD/DDPC/EDTA and PN159.

### Example 3

### In Vivo Permeation Enhancement by PN159 for a Peptide Hormone Therapeutic Agent Equals or Exceeds That of Small Molecule Permeation Enhancers

20 male New Zealand White rabbits age 3-6 months and weighing 2.1-3.0 kg were randomly assigned into one of 5 treatment groups with four animals per group. Test animals were dosed at 15 μl/kg and intranasally via pipette. Table 4 below indicates the composition of five different dose groups.

For dosing group 1 (see Table 1) a clinical formulation of PYY including small molecule permeation enhancers was used. The small molecule enhancers in these studies included methyl-βcyclodextrin, phosphatidylcholine didecanoyl (DDPC), and/or EDTA. Dosing group 2 received PYY dissolved in phosphate buffered saline (PBS). For dosing groups 3-5, various concentrations of PN 159 were added to dosing group 2, so that each of dosing groups 3-5 consisted of PYY, PN159, and PBS.

TABLE 1

| | | | | | PYY |
|---|---|---|---|---|---|
| | | Permeation | Dose Conc | Dose Vol | Dose |
| Group | Animals | enhancers | (mg/ml) | (ml/kg) | (μg/kg) |
| 1 | 4M | Small molecule | 13.67 | 0.015 | 205 |
| | | permeation | | | |
| | | enhancers | | | |
| 2 | 4M | None | 13.67 | 0.015 | 205 |
| 3 | 4M | 25 μM PN159 | 13.67 | 0.015 | 205 |
| 4 | 4M | 50 μM PN159 | 13.67 | 0.015 | 205 |
| 5 | 4M | 100 μM PN159 | 13.67 | 0.015 | 205 |

Serial blood samples (about 2 ml each) were collected by direct venipuncture from a marginal ear vein into blood collection tubes containing EDTA as an anticoagulant. Blood samples were collected at 0, 2.5, 5, 10, 15, 30, 45, 60, and 120 minutes post-dosing. After collection of the blood, the tubes were gently rocked several times for anti-coagulation, and then 50 μl aprotinin solution was added. The blood was centrifuged at approximately 1,600×g for 15 minutes at approximately 4° C., and plasma samples were dispensed into duplicate aliquots and stored frozen at approximately −70° C.

Averaging all four animals in a treatment group, the following plasma concentrations of PYY were measured (Table 2):

TABLE 2

| | Group 1 | | | | |
|---|---|---|---|---|---|
| | Small | Group 2 | | | |
| | molecule | No | Group 3 | Group 4 | Group 5 |
| Time, | permeation | permeation | 25 μM | 50 μM | 100 μM |
| mins | enhancers | enhancers | PN159 | PN159 | PN159 |
| 0 | 183.825 | 257.3 | 228.675 | 424.4 | 294.225 |
| 2.5 | 1280.7 | 242.8 | 526.375 | 749.975 | 1748.225 |
| 5 | 1449.425 | 273.675 | 1430.15 | 1293.4 | 3088.2 |
| 10 | 8251.8 | 372.05 | 6521.7 | 12517.2 | 14486.6 |
| 15 | 13731.2 | 398.225 | 12563.075 | 34455.3 | 20882.725 |
| 30 | 19537.55 | 476.475 | 15222.6 | 35294.375 | 25470.475 |
| 45 | 13036.075 | 340.7 | 9081.125 | 21582.225 | 16499.55 |
| 60 | 7080.875 | 283.825 | 4843.15 | 9461.925 | 10676.625 |
| 120 | 1671.9 | 192.575 | 1224.2 | 2337.775 | 1891.275 |

The pharmacokinetic data calculated from the above data is shown below in Table 6:

TABLE 3

| Variable | Group | Mean | SD | SE |
|---|---|---|---|---|
| Cmax (pg/mL) | 1 | 19832.18 | 17737.21 | 8868.605 |
| Tmax (min) | 1 | 32.5 | 20.6155 | 10.3078 |
| AUClast | 1 | 991732.1 | 930296.3 | 465148.1 |
| (min*pg/mL) | | | | |
| AUCINF | 1 | 1357132 | 928368.5 | 535993.8 |
| (min*pg/mL) | | | | |
| t½ (min) | 1 | 23.69 | 1.713 | 0.989 |
| Cmax (pg/mL) | 2 | 516.725 | 196.492 | 98.246 |
| Tmax (min) | 2 | 26.25 | 14.3614 | 7.1807 |
| AUClast | 2 | 36475.72 | 9926.104 | 4963.052 |
| (min*pg/mL) | | | | |
| AUCINF | 2 | 60847.41 | 17688.31 | 8844.156 |
| (min*pg/mL) | | | | |
| t½ (min) | 2 | 84.5919 | 26.8859 | 13.4429 |
| Cmax (pg/mL) | 3 | 15533.95 | 13225.88 | 6612.941 |
| Tmax (min) | 3 | 22.5 | 8.6603 | 4.3301 |
| AUClast | 3 | 748104.1 | 661213.8 | 330606.9 |
| (min*pg/mL) | | | | |
| AUCINF | 3 | 796354.7 | 721017.8 | 360508.9 |
| (min*pg/mL) | | | | |
| t½ (min) | 3 | 24.8467 | 4.3108 | 2.1554 |
| Cmax (pg/mL) | 4 | 40995.53 | 32112.71 | 16056.35 |
| Tmax (min) | 4 | 26.25 | 7.5 | 3.75 |
| AUClast | 4 | 1692499 | 1339896 | 669947.8 |
| (min*pg/mL) | | | | |
| AUCINF | 4 | 1787348 | 1395185 | 697592.4 |
| (min*pg/mL) | | | | |
| t½ (min) | 4 | 25.5355 | 8.6139 | 4.3069 |
| Cmax (pg/mL) | 5 | 27974.4 | 17584.31 | 8792.154 |
| Tmax (min) | 5 | 33.75 | 18.8746 | 9.4373 |
| AUClast | 5 | 1384241 | 817758.8 | 408879.4 |
| (min*pg/mL) | | | | |
| AUCINF | 5 | 1518949 | 1030623 | 595030.3 |
| (min*pg/mL) | | | | |
| t½ (min) | 5 | 20.4628 | 6.5069 | 3.7568 |

Compared with the Group 2 (no enhancer) formulation, the following relative enhancement ratios were determined (Table 4):

TABLE 4

| | | Relative | Relative |
|---|---|---|---|
| Group | Formulation | Cmax | AUC last |
| 1 | Small molecule permeation enhancers | 38x | 27x |
| 3 | PN159, 25 μm | 30x | 21x |
| 4 | PN159, 50 μm | 79x | 46x |
| 5 | PN159, 100 μm | 54x | 38x |

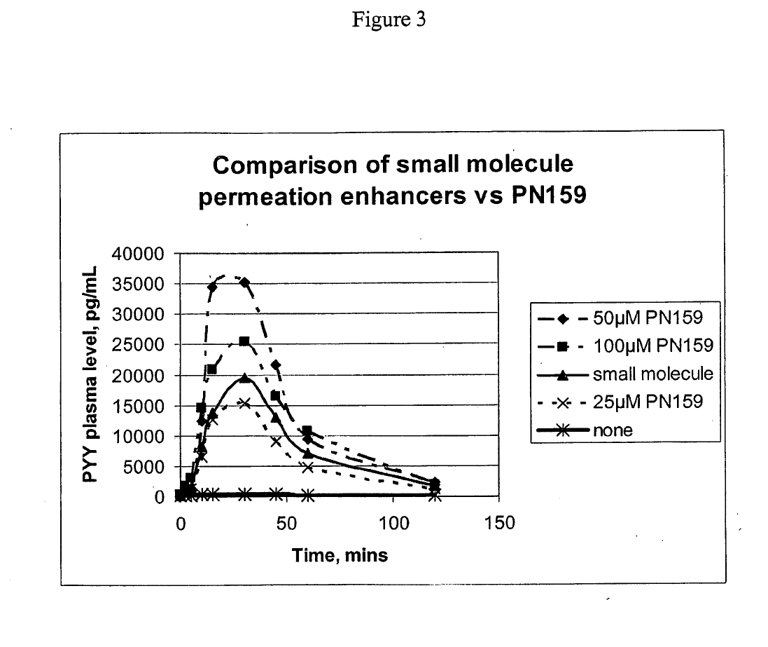

The foregoing data are graphically depicted in FIG. 3, and demonstrate that permeabilizing peptides of the invention, as exemplified by PN159, are able to enhance in vivo intranasal permeation of a human hormone peptide thereapeutic to an equal or greater degree compared to small molecule permeation enhancers. The greatest effect of the peptide is seen at a 50 μM concentration. The 100 μM concentration resulted in somewhat less permeation, although both resulted in higher permeation than the small molecule permeation enhancers.

### Example 4

### Permeation Enhancement by PN159 For an Oligoeptide Therapeutic Agent

The present example demonstrates efficacy of an exemplary peptide of the invention, PN159 to enhance epithelial permeation for a cyclic pentapeptide, melanocortin-4 receptor agonist (MC-4RA) a model oligopeptide agonist for a mammalian cellular receptor. In this example, a combination of one or more of the permeabilizing peptides with MC-4RA is described. Useful formulations in this context can include a combination of an oligopeptide therapeutic, a permeabilizing peptide, and one or more other permeation enhancers. The formulation may also contain buffers, tonicifying agents, pH adjustment agents, and peptide/protein stabilizers such as amino acids, sugars or polyols, polymers, and salts.

The effect of PN159 on permeation of MC-4RA was evaluated in this study. MC-4RA was a methanesulphonate salt with a molecular weight of approximately 1,100 Da, which modulates activity of the MC-4 receptor. The PN159 concentrations evaluated are 5, 25, 50, and 100 μM. 45 mg/ml M-β-CD was used as a solubilizer for all formulations to achieve 10 mg/ml peptide concentration. The effect of PN159 was assessed either by itself or in combination with EDTA (1, 2.5, 5, or 10 mg/ml). The formulation pH was fixed at 4 and the osmolarity was at 220 mOsm/kg.

HPLC Method

The concentrations of MC-4RA in the basolateral media was analyzed by the RP-HPLC using a C18 RP chromatography with a flow rate of 1 mL/minute and a column temperature of 25° C.
Solvent A: 0.1% TFA in water; Solvent B: 0.1% TFA in ACN
Injection Volume: 50 μL
Detection: 220 nm
Run Time: 15 min

MC-4RA was combined with 5, 25, 50, and 100 μM PN159, pH 4 and osmolarity ˜220 mOsm/kg. The combination was tested using an in vitro epithelial tissue model to monitor PTH permeation, transepithelial electrical resistance (TER), and the cytotoxicity of the formulation by MTT and LDH assays.

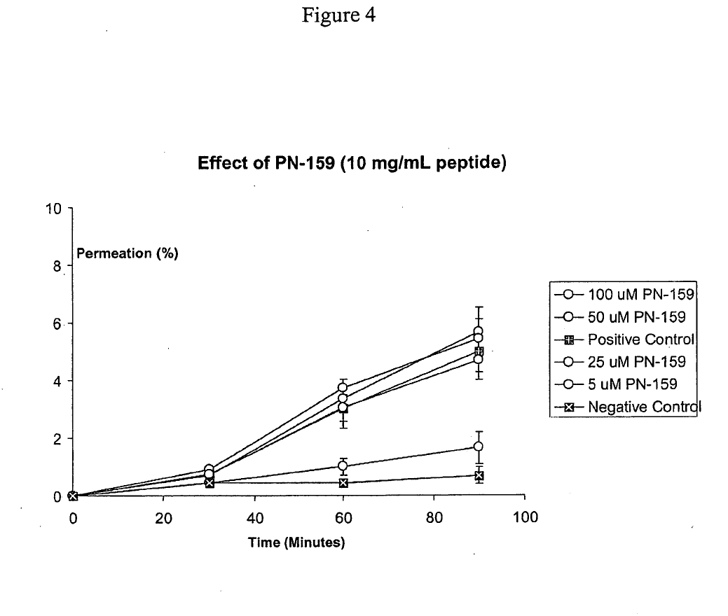

The results of studies of the permeation of MC-4RA are shown in FIG. 4. These studies evince that PN159, in addition to enhancing mucosal permeation for peptide hormone therapeutics, also significantly enhance epithelial permeation for oligopeptide therapeutic agents.

### Example 5

### Permeation Enhancement by PN159 for a Small Molecule Drug

The present example demonstrates efficacy of an exemplary peptide of the invention, PN159 to enhance epithelial permeation for a small molecule drug, exemplified by the acetylcholinesterase (ACE) inhibitor galantamine. In this example, a combination of one or more of the permeabilizing peptides with a small molecule drug is described. Useful formulations in this context can include a combination of a small molecule drug, a permeabilizing peptide, and one or more other permeation enhancers. The formulation may also contain buffers, tonicifying agents, pH adjustment agents, stabilizers and/or preservatives.

The present invention combines galantamine with PN159 to enhance permeation of galantamine across the nasal mucosa. This increase in drug permeation is unexpected because galantamine is a small molecule that can permeate the nasal epithial membrane independently. The significant enhancement of galantamine permeation across epithelia mediated by addition of excipients which enhance the permeation of peptides is therefore surprising, on the basis that such excipients would not ordinarily be expected to significantly increase permeation of galantamine across the epithelial tissue layer. The invention therefore will facilitate nasal delivery of galantamine and other small molecule drugs by increasing their bioavailability.

In the present studies, 40 mg/ml galantamine in the lactate salt form was combined with 25, 50, and 100 μM PN159 in solution, pH 5.0 and osmolarity ˜270 mOsm. The combination was tested using an in vitro epithial tissue model to monitor galantamine permeation, transepithelial electrical resistance (TER), and the cytotoxicity of the formulation by LDH and MTT assays as described above. Permeation measurements for galantamine were conducted by standard HPLC analysis, as follows.

HPLC Analysis

Galantamine concentration in the formulation and in the basolateral media (permeation samples) was determined using an isocratic LC (Waters Alliance) method with UV detection.
Column: Waters Symmetry Shield, C18, 5 um, 25×0.46 cm
Mobile phase: 5% ACN in 50 mM ammonium formate, pH 3.0
Flow rate: 1 ml/min
Column temperature: 30° C.
Calibration curve: 0-400 μg/ml Galantamine HBr
Detection: UV at 285 nm

Based on the foregoing studies, PN159 improves transmucosal delivery of small molecules. Galantamine was chosen as a model low molecular weight drug, and the results for this molecule are considered predictive of permeabilizing peptide activity for other small molecule drugs. To evaluate permeabilizing activity in this context, 40 mg/ml galantamine in the lactate salt form was combined with 25, 50, and 100 μM PN159 in solution, pH 5.0 and osmolarity ˜270 mOsm. The combination was tested using an in vitro epithelal tissue model to monitor galantamine permeation, transepithelial electrical resistance (TER), and the cytotoxicity of the formulation by LDH and MTT assays.

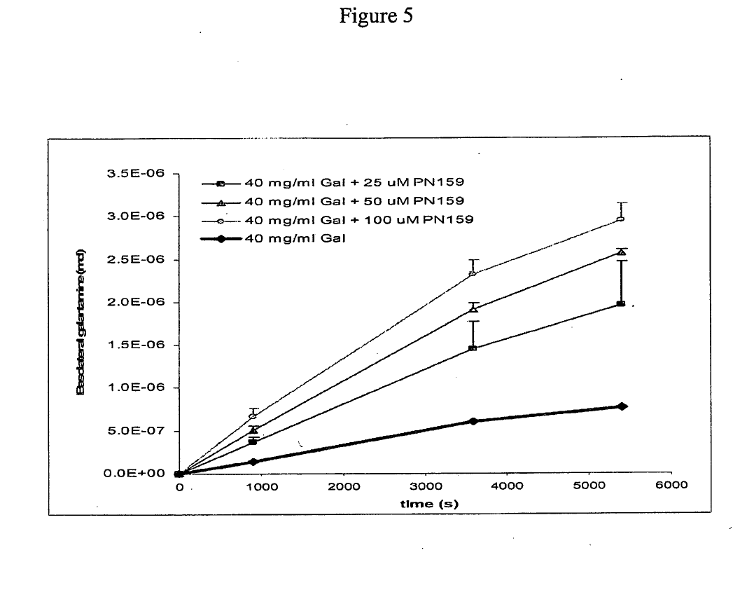

In the in vitro tissue model, the addition of PN159 resulted in a dramatic increase in drug permeation across the cell barrier. Specifically, there was a 2.5-3.5 fold increase in the Pₐₚₚ of 40 mg/ml galantamine. (FIG. 5)

PN159 reduced TER in the presence of galantamine just as described in Example II.

Cell viability remained high (>80%) in the presence of galantamine lactate and PN159 at all concentrations tested. Conversely, cyctotoxicity was low in the presence of PN159 and galantamine lactate, as measured by LDH. Both of these assays suggest that PN159 is not toxic to the epithelial membrane.

Summarizing the foregoing results, PN159 has been demonstrated herein to surprisingly increase epithelial permeation of galantamine as a model low molecular weight drug. The addition of PN159 to galantamine in solution significantly enhances galantamine permeation across epithelial monolayers. Evidence shows that PN159 temporarily reduces TER across the epithelial membrane without damaging the cells in the membrane, as measured by high cell viability and low cytotoxicity. PN159 therefore is an exemplary peptide for enhancing bioavailability of galantamine and other small molecule druges in vivo, via the same mechanism that is demonstrated herein using in vitro models. It is further expected that PN159 will enhance permeation of galantamine at higher concentrations as well.

Although the foregoing invention has been described in detail by way of example for purposes of clarity of understanding, it will be apparent to the artisan that certain changes and modifications may be practiced within the scope of the appended claims which are presented by way of illustration not limitation. In this context, various publications and other references have been cited within the foregoing disclosure for economy of description. Each of these references is incorporated herein by reference in its entirety for all purposes. It is noted, however, that the various publications discussed herein are incorporated solely for their disclosure prior to the filing date of the present application, and the inventors reserve the right to antedate such disclosure by virtue of prior invention.

Chemical Stability

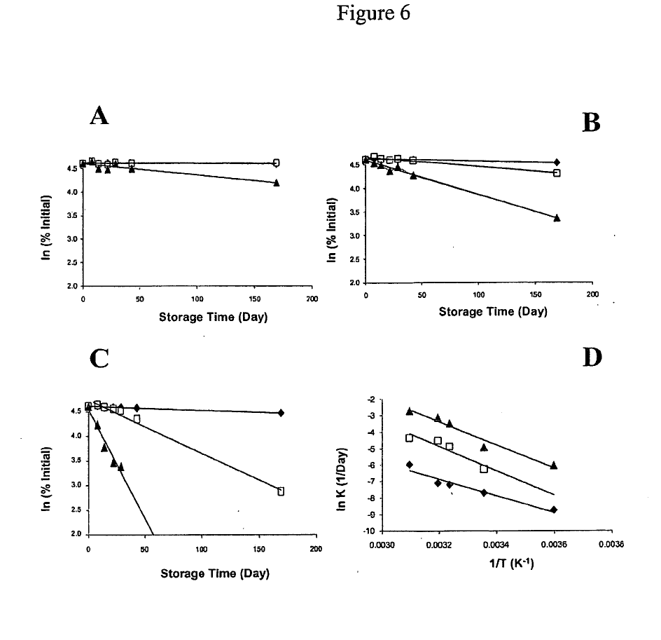

The chemical stability of the PN159 was determined under therapeutically relevant storage conditions. A stability indicating HPLC method was employed. Solutions (50 mM) were stored at various pH (4.0, 7.3, and 9.0) and temperature (5° C., 25° C., 35° C., 40° C., and 50° C.) conditions. Samples at pH 4 contained 10 mM citrate buffer. Samples at pH 7.3 and 9.0 contained 10 mM phosphate buffer. Representative storage stability data (including the Arrhenius plot) are depicted in FIG. 6. As can be seen, the PN159 was most chemically stable at low temperature and pH. For example, at 5° C. and pH 4.0 or pH7.3, there was essentially 100% recovery of PN159 for six month storage. When the storage temperature was increased to 25° C., there was a 7% and 26% loss of native PN159 for samples at pH 4 or pH 7, respectively, after six months. At pH 9 and/or at elevated temperature, e.g., 40 to 50° C., rapid deterioration of the PN159 ensued. The pH range of 4.0 to 7.3 and the temperature range of refrigerated to ambient are most relevant for intranasal formulations. Therefore, these data support that the PN159 can maintain chemical integrity under storage conditions relevant to IN formulations.

There was a marked increase in rate of drug permeated vs. time. These data were used to calculate the permeability constant (Pₐₚₚ), presented in Table 5.

TABLE 5
Pₐₚₚ measured using the in vitro tissue model.

| | [PN159] | Papp | |
|---|---|---|---|
| Drug Formulation | (μM) | (cm/s) | Relative Pₐₚₚ |
| Galantamine | 0 | 2.1 × 10⁻⁶ | 1.0 |
| 40 mg/mL, pH 5.0 | 25 | 5.1 × 10⁻⁶ | 2.4 |
| | 50 | 6.2 × 10⁻⁶ | 3.0 |
| | 100 | 7.2 × 10⁻⁶ | 3.4 |
| Calcitonin | 0 | 9.7 × 10⁻⁸ | 1.0 |
| 1 mg/mL, pH 3.5 | 25 | 2.2 × 10⁻⁶ | 23. |
| | 50 | 3.3 × 10⁻⁶ | 34. |
| | 100 | 4.6 × 10⁻⁶ | 47. |
| PTH₁₋₃₄ | 0 | 1.1 × 10⁻⁷ | 1.0 |
| 1 mg/mL, pH 4.5 | 25 | 3.4 × 10⁻⁷ | 3.0 |
| | 50 | 4.9 × 10⁻⁷ | 4.5 |
| | 100 | 4.3 × 10⁻⁷ | 3.9 |
| PYY₃₋₃₆ | 0^{a} | 1.3 × 10⁻⁷ | 1.0 |
| 1 mg/mL, pH 7.0 | 25 | 1.6 × 10⁻⁶ | 12. |
| | 100 | 2.2 × 10⁻⁶ | 17. |

^{a}pH was 5.0

In the absence of PN159, the Pₐₚₚ for galantamine was about 2.1×10⁻⁶ cm/s. In the presence of 25, 50 and 100 mM PN159, Pₐₚₚ was 5.1×10⁻⁶, 6.2×10⁻⁶, and 7.2×10⁻⁶ cm/s, respectively. Thus, the PN159 afforded a 2.4- to 3.4-fold increase in Pₐₚₚ of this model low-molecular-weight drug.

Having established the utility of the PN159 for transmucosal formulations of low-molecular-weight compounds, it was important to discern whether these observations could be extrapolated to larger molecules, e.g., therapeutic peptides and proteins. For this purpose, in vitro tissue studies were performed on salmon calcitonin as a model therapeutic peptide in the absence and presence of 25, 50, and 100 mM PN159. In the absence of PN159, the Pₐₚₚ for calcitonin was about 1×10⁻⁷ cm/s, about an order of magnitude lower than that for galantamine, presumably due to the difference in molecular weight. The data reveal a dramatic increased in calcitonin permeation in the presence of the PN159, up to a 23- to 47-fold increase in Pₐₚₚ compared to the case of the calcitonin alone (Table 6).

In order to explore the generality of these findings, two additional peptides, namely human parathyroid hormone 1-34 (PTH₁₋₃₄) and human peptide YY 3-36 (PYY₃₋₃₆) were examined in the in vitro model in the absence and presence of PN159 (Pₐₚₚ data presented in Table 6). In the absence of PN159, the Pₐₚₚ of these two peptides was consistent to that for calcitonin. In the case of PTH₁₋₃₄, the presence of PN159 afforded about 3-5 fold increase in Pₐₚₚ. When PYY₃₋₃₆ was formulated in the presence of PN159, the Pₐₚₚ was increased about 12- to 17-fold. These data confirm the generality of our finding that the PN159 has utility for enhancing transmucosal drug delivery.

### Example 6

### D-amino Acid Versions of PN159

The D-amino acid substituted PN159 peptides listed in Table 6 were synthesized and purified, and were tested for their ability to enhance TER and permeability, using the methods described in the Examples above.

TABLE 6
D-amino acid substitutions

| | | | | Perm(x)/Perm |
|---|---|---|---|---|
| | | | TER(x)/TER | |
| Peptide | Sequence | Description | (159) +/− SEM | (159 +/− SEM |
| PN159 | NH2-KLALKLALKALKAALKLA-amide | model | 1.00 +/− 0.14 | 1.00 +/− 0.13 |
| | (SEQ ID NO: 1) | amphipathic | | |
| | | peptide | | |
| PN393 | NH2-klalklalkalkaalkla-amide | All D- | 1.06 +/− 0.00 | 1.02 +/− 0.16 |
| | (SEQ ID NO: 2) | substituted | | |
| PN407 | NH2-LKlLKkLlkKLLkLL-amide | Leucine and | 1.08 +/− 0.01 | 1.20 +/− 0.05 |
| | (SEQ ID NO: 3) | Lysine rich | | |
| | | with D-subs | | |
| PN434 | NH2-KLaLKlALkAlkAALkLA-amide | D-substituted | 0.12 +/− 0.01 | 0.02 +/− 0.00 |
| | (SEQ ID NO: 4) | | | |
| PN408 | NH2-alklaaklaklalklalk-amide | PN159 retro- | 1.05 +/− 0.01 | 1.16 +/− 0.07 |
| | (SEQ ID NO: 5) | inverso | | |

PN407 shows minor but statistically significant improvement on permeability. Both All D and retro inverso forms of PN159 show decreased TER recovery suggesting a longer TER reduction effect that might be useful for in vivo delivery. Random D substitution (PN434) can cause null activities both on TER reduction and permeability enchancement.

### Example 7

### PN159 Length Chances

PN159 peptides having length changes listed in Table 7 were synthesized and purified, and were tested for their ability to enhance TER and permeability, using the methods described in the Examples above.

| | | | TER(x)/TER | Perm(x)/Perm |
|---|---|---|---|---|
| Peptide | Sequence | Description | (159) +/− SEM* | (159) +/− SEM* |
| PN159 | NH2-KLALKLALKALKAALKLA-amide | model peptide | 1.00 +/− 0.14 | 1.00 +/− 0.13 |
| | (SEQ ID NO: 1) | | | |
| PN417 | NH2-KLALKLALKALKAA-amide | Shortened 14 aa | 0.19 +/− 0.01 | 0.04 +/− 0.01 |
| | (SEQ ID NO: 6) | | | |
| PN418 | NH2-KLALKLALKALKAALK-amide | Shortened 16 aa | 1.05 +/− 0.05 | 0.64 +/− 0.08 |
| | (SEQ ID NO: 7) | | | |
| PN419 | NH2-KLALKLALKALKAALKLALK-amide | Lengthened 20 aa | 1.23 +/− 0.01 | 0.74 +/− 0.13 |
| | (SEQ ID NO: 8) | | | |
| PN420 | NH2-KLALKLALKALKAALKLALKLA-amide | Lengthened 22 aa | 0.77 +/− 0.05 | 0.24 +/− 0.05 |
| | (SEQ ID NO: 9) | | | |
| PN421 | NH2-KLALKLALKALKAALKLALKLALK-amide | Lengthened 24 aa | 0.74 +/− 0.11 | 0.17 +/− 0.06 |
| | (SEQ ID NO: 10) | | | |
| PN422 | NH2-KLALKLALKALKAALKLALKLALKAL-amide | Lengthened 26 aa | 0.47 +/− 0.07 | 0.07 +/− 0.01 |
| | (SEQ ID NO: 11) | | | |

mean values from multiple repeats

The results show that lengths of PN159 is important for its TER reduction and enhanced permeability activity. Lengthen PN159 to 20 aa increased TER reduction effect but reduced permeability effect. TER recovery is slower. Shorten PN159 to 16 aa show no effect on TER reduction but reduced permeability effect. Shorten PN159 to 14 aa drastically reduced permeability, suggesting the length of PN159 is crucial of permeability. Contrary to the permeability effect, the effect of the PN159 length on TER reduction is more gradual.

### Example 8

### Tryptophan and Arginine Substitutions in PN159

PN159 peptides having amino acid substitutions listed in Table 8 were synthesized and purified, and were tested for their ability to enhance TER and permeability, using the methods described in the Examples above.

TABLE 8
Amino Acid Substitutions

| | | | | |
|---|---|---|---|---|
| | | | Relative | |
| | | | TER | Relative |
| Peptide | Sequence | Name | Decrease | Permeability |

| | | | | |
|---|---|---|---|---|
| PN159 | NH2-KLALKLALKALKAALKLA-amide | model peptide | 1.0 | 1.0 |
| | (SEQ ID NO: 1) | | | |
| PN394 | NH2-RLALRLALRALRAALRLA-amide | Argenine | 0.7 | 0.1 |
| | (SEQ ID NO: 12) | | | |
| PN395 | NH2-RLAWRLALRALRAALRLA-amide | Argenine and | 0.8 | 0.2 |
| | (SEQ ID NO: 13) | Single | | |
| | | Tryptophan | | |
| PN0425 | NH2-KLAWKLALKALKAALKLA-amide | Single | 1.0 | 1.2 |
| | (SEQ ID NO: 14) | Tryptophan | | |
| PN0427 | NH2-KLAWKLALKALKAAWKLA-amide | Two Tryptophan | 1.0 | 1.0 |
| | (SEQ ID NO: 15) | | | |
| PN0428 | NH2-KLAWKLAWKALKAAWKLA-amide | Three | 0.7 | 1.0 |
| | (SEQ ID NO: 16) | Tryptophan | | |
| PN406 | NH2-LKLLKKLLKKLLKLL-amide | Leucine and | 0.9 | 0.6 |
| | (SEQ ID NO: 17) | Lysine rich | | |
| PN407 | NH2-LKlLKkLlkKLLkLL-amide | Leucine and | 1.1 | 1.2 |
| | (SEQ ID NO: 18) | Lysine rich with | | |
| | | D-subs | | |
| PN443 | NH2-LKTLATALTKLAKTLTTL-amide | Threonine | 0.3 | 0.1 |
| | (SEQ ID NO: 19) | | | |
| PN448 | NH2-KLALKLALKNLKAALKLA-amide | Asparagine | 0.4 | 0.0 |
| | (SEQ ID NO: 24) | | | |

The results show that an arginine guanidinium headgroup is more effective than lysine and histidine. Tryptophan is preferential amino acid at the water-membrane interface1. PN407 shows minor but statistically significant improvement on permeability. Arginine replacement of Lysine drastically reduce the permeability but has less impact on TER reduction, suggesting the importance of Lysine is permeability. Single replacement of Alanine on aa10 with Asparagine abolish permeability, suggesting the important of alpha helicy for PN159 activities.

### Example 9

### Hydrophobicity Chances in PN159

PN159 peptides having amino acid substitutions listed in Table 9 were synthesized and purified, and were tested for their ability to enhance TER and permeability, using the methods described in the Examples above.

TABLE 9
Hydrophobic Faces

| | | | TER(x)/TER | Perm(x)/Perm |
|---|---|---|---|---|
| Peptide | Sequence | Description | (159) +/− SEM* | (159) +/− SEM* |
| PN159 | NH2-KLALKLALKALKAALKLA-amide | model peptide | 1.00 +/− 0.14 | 1.00 +/− 0.13 |
| | (SEQ ID NO: 1) | | | |
| PN424 | NH2-KALKLKAALALLAKLKLA-amide | non-amphipathic | 0.59 +/− 0.07 | 0.20 +/− 0.04 |
| | (SEQ ID NO: 20) | | | |
| PN441 | NH2-KLAAALLKKAKKLAAALL-amide | 200° | 0.54 +/− 0.04 | 0.35 +/− 0.04 |
| | (SEQ ID NO: 21) | hydrophobic | | |
| | | face | | |
| PN442 | NH2-KALAALLKKAAKLLAALK-amide | 180° face | 0.93 +/− 0.03 | 0.81 +/− 0.03 |
| | (SEQ ID NO: 22) | | | |
| PN444 | NH2-KALAALLKKLAKLLAALK-amide | 180° face | 0.82 +/− 0.05 | 0.41 +/− 0.08 |
| | (SEQ ID NO: 23) | | | |

* mean values from multiple repeats

PN159 has 280 degrees of hydrophobic faces. The results show that reduction of the hydrophobic faces can cause reduction of PN159 activities. Amphipathicity of PN159 is also important for its activities.

What is claimed is:

1. An isolated peptide comprising a sequence selected from the group consisting of SEQ ID NOs:1 and 20-23 lengthened by one or more amino acid residues, and pharmaceutically-acceptable salts thereof.

2. The peptide of claim 1, the peptide comprising a sequence selected from the group consisting of SEQ ID NOs:8-11.

3. The peptide of claim 1, wherein the peptide is covalently linked to a polyethyleneglycol chain having a molecular size less than 40 kDa.

4. A composition comprising the peptide of claim 1 and an active agent selected from the group consisting of GLP-1, PYY3-36, PTH1-34, Exendin-4, beta-interferon, alpha-interferon, insulin, erythropoietin, G-CSF, GM-CSF, human growth hormone, and analogs thereof.

5. A composition comprising the peptide of claim 1 and an active agent selected from the group consisting of a peptide, a protein, a nucleic acid, a double-stranded RNA, a hematopoietic, an antiinfective; an antidementia; an antiviral, an antitumoral, an antipyretic, an analgesic, an anti-inflammatory, an antiulcerative, an antiallergenic, an antidepressant, a psychotropic, a cardiotonic, an antiarrythmic, a vasodilator, an antihypertensive, a hypotensive diuretic, an antidiabetic, an anticoagulant, a cholesterol-lowering agent, a therapeutic for osteoporosis, a hormone, an antibiotic, a vaccine, a cytokine, a hormone, a growth factor, a cardiovascular factor, a cell adhesion factor, a central or peripheral nervous system factor, a humoral electrolyte factor, a hemal organic substance, a bone growth factor, a gastrointestinal factor, a kidney factor, a connective tissue factor, a sense organ factor, an immune system factor, a respiratory system factor, a genital organ factor, an androgen, an estrogen, a prostaglandin, a somatotropin, a gonadotropin, an interleukin, a steroid, a bacterial toxoid, hirugen, hirulos, hirudine, a monoclonal antibody, a polyclonal antibody, a humanized antibody, an antibody fragment, an immunoglobin, morphine, hydromorphone, oxymorphone, lovorphanol, levallorphan, codeine, nalmefene, nalorphine, nalozone, naltrexone, buprenorphine, butorphanol, or nalbufine, cortisone, hydrocortisone, fludrocortisone, prednisone, prednisolone, methylprednisolone, triamcinolone, dexamethoasone, betamethoasone, paramethosone, fluocinolone, colchicine, acetaminophen, a non-steroidal anti-inflammatory agent NSAID, acyclovir, ribavarin, trifluorothyridine, Ara-A Arabinofuranosyladenine, acylguanosine, nordeoxyguanosine, azidothymidine, dideoxyadenosine, dideoxycytidine, spironolactone, testosterone, estradiol, progestin, gonadotrophin, estrogen, progesterone, papaverine, nitroglycerin, a vasoactive intestinal peptide, calcitonin gene-related peptide, cyproheptadine, doxepin, imipramine, cimetidine, dextromethorphan, clozaril, superoxide dismutase, neuroenkephalinase, amphotericin B, griseofulvin, miconazole, ketoconazole, tioconazol, itraconazole, fluconazole, cephalosporin, tetracycline, aminoglucoside, erythromicin, gentamicin, polymyxin B, 5-fluorouracil, bleomycin, methotrexate, hydroxyurea, dideoxyinosine, floxuridine, 6-mercaptopurine, doxorubicin, daunorubicin, 1-darubicin, taxol, paclitaxel, tocopherol, quinidine, prazosin, verapamil, nifedipine, diltiazem, tissue plasminogen activator TPA, epidermal growth factor EGF, fibroblast growth factor FGF-acidic or basic, platelet derived growth factor PDGF, transforming growth factor TGF-alpha or beta, vasoactive intestinal peptide, tumor necrosis factor TNF, hypothalmic releasing factor, prolactin, thyroid stimulating hormone TSH, adrenocorticotropic hormone ACTH, parathyroid hormone PTH, follicle stimulating hormone FSF, luteinizing hormone releasing hormone LHRH, endorphin, glucagon, calcitonin, oxytocin, carbetocin, aldoetecone, enkaphalin, somatostin, somatotropin, somatomedin, alpha-melanocyte stimulating hormone, lidocaine, sufentainil, terbutaline, droperidol, scopolamine, gonadorelin, ciclopirox, buspirone, cromolyn sodium, midazolam, cyclosporin, lisinopril, captopril, delapril, ranitidine, famotidine, superoxide dismutase, asparaginase, arginase, arginine deaminease, adenosine deaminase ribonuclease, trypsin, chemotrypsin, papain, bombesin, substance P, vasopressin, alpha-globulins, transferrin, fibrinogen, beta-lipoprotein, beta-globulin, prothrombin, ceruloplasmin, alpha2-glycoprotein, alpha2-globulin, fetuin, alpha1-lipoprotein, alpha1-globulin, albumin, and prealbumin.

6. An isolated peptide comprising a sequence selected from the group consisting of
SEQ ID NOs:1 and 20-23, and variants thereof, having 14-26 amino acid residues, wherein the peptide has at least two D-amino acid residues, and pharmaceutically-acceptable salts thereof.

7. The peptide of claim 6, the peptide comprising the sequence SEQ ID NOs:2-5.

8. The peptide of claim 6, wherein the peptide is covalently linked to a polyethyleneglycol chain having a molecular size less than 40 kDa.

9. A composition comprising the peptide of claim 6 and an active agent selected from the group consisting of GLP-1, PYY3-36, PTH1-34, Exendin-4, beta-interferon, alpha-interferon, insulin, erythropoietin, G-CSF, GM-CSF, human growth hormone, and analogs thereof.

10. A composition comprising the peptide of claim 6 and an active agent selected from the group consisting of a peptide, a protein, a nucleic acid, a double-stranded RNA, a hematopoietic, an antiinfective; an antidementia; an antiviral, an antitumoral, an antipyretic, an analgesic, an anti-inflammatory, an antiulcerative, an antiallergenic, an antidepressant, a psychotropic, a cardiotonic, an antiarrythmic, a vasodilator, an antihypertensive, a hypotensive diuretic, an antidiabetic, an anticoagulant, a cholesterol-lowering agent, a therapeutic for osteoporosis, a hormone, an antibiotic, a vaccine, a cytokine, a hormone, a growth factor, a cardiovascular factor, a cell adhesion factor, a central or peripheral nervous system factor, a humoral electrolyte factor, a hemal organic substance, a bone growth factor, a gastrointestinal factor, a kidney factor, a connective tissue factor, a sense organ factor, an immune system factor, a respiratory system factor, a genital organ factor, an androgen, an estrogen, a prostaglandin, a somatotropin, a gonadotropin, an interleukin, a steroid, a bacterial toxoid, hirugen, hirulos, hirudine, a monoclonal antibody, a polyclonal antibody, a humanized antibody, an antibody fragment, an immunoglobin, morphine, hydromorphone, oxymorphone, lovorphanol, levallorphan, codeine, nalmefene, nalorphine, nalozone, naltrexone, buprenorphine, butorphanol, or nalbufine, cortisone, hydrocortisone, fludrocortisone, prednisone, prednisolone, methylprednisolone, triamcinolone, dexamethoasone, betamethoasone, paramethosone, fluocinolone, colchicine, acetaminophen, a non-steroidal anti-inflammatory agent NSAID, acyclovir, ribavarin, trifluorothyridine, Ara-A Arabinofuranosyladenine, acylguanosine, nordeoxyguanosine, azidothymidine, dideoxyadenosine, dideoxycytidine, spironolactone, testosterone, estradiol, progestin, gonadotrophin, estrogen, progesterone, papaverine, nitroglycerin, a vasoactive intestinal peptide, calcitonin gene-related peptide, cyproheptadine, doxepin, imipramine, cimetidine, dextromethorphan, clozaril, superoxide dismutase, neuroenkephalinase, amphotericin B, griseofulvin, miconazole, ketoconazole, tioconazol, itraconazole, fluconazole, cephalosporin, tetracycline, aminoglucoside, erythromicin, gentamicin, polymyxin B, 5-fluorouracil, bleomycin, methotrexate, hydroxyurea, dideoxyinosine, floxuridine, 6-mercaptopurine, doxorubicin, daunorubicin, 1-darubicin, taxol, paclitaxel, tocopherol, quinidine, prazosin, verapamil, nifedipine, diltiazem, tissue plasminogen activator TPA, epidermal growth factor EGF, fibroblast growth factor FGF-acidic or basic, platelet derived growth factor PDGF, transforming growth factor TGF-alpha or beta, vasoactive intestinal peptide, tumor necrosis factor TNF, hypothalmic releasing factor, prolactin, thyroid stimulating hormone TSH, adrenocorticotropic hormone ACTH, parathyroid hormone PTH, follicle stimulating hormone FSF, luteinizing hormone releasing hormone LHRH, endorphin, glucagon, calcitonin, oxytocin, carbetocin, aldoetecone, enkaphalin, somatostin, somatotropin, somatomedin, alpha-melanocyte stimulating hormone, lidocaine, sufentainil, terbutaline, droperidol, scopolamine, gonadorelin, ciclopirox, buspirone, cromolyn sodium, midazolam, cyclosporin, lisinopril, captopril, delapril, ranitidine, famotidine, superoxide dismutase, asparaginase, arginase, arginine deaminease, adenosine deaminase ribonuclease, trypsin, chemotrypsin, papain, bombesin, substance P, vasopressin, alpha-globulins, transferrin, fibrinogen, beta-lipoprotein, beta-globulin, prothrombin, ceruloplasmin, alpha2-glycoprotein, alpha2-globulin, fetuin, alpha1-lipoprotein, alpha1-globulin, albumin, and prealbumin.

11. An isolated peptide comprising the retro-inverso of a sequence selected from the group consisting of SEQ ID NOs:1 and 20-23, and variants thereof, having 14-26 amino acid residues, and pharmaceutically-acceptable salts thereof.

12. The peptide of claim 11, wherein the peptide has at least two D-amino acid residues.

13. The peptide of claim 11, the peptide comprising the sequence SEQ ID NO:5.

14. A composition comprising the peptide of claim 11 and an active agent selected from the group consisting of GLP-1, PYY3-36, PTH1-34, Exendin-4, beta-interferon, alpha-interferon, insulin, erythropoietin, G-CSF, GM-CSF, human growth hormone, and analogs thereof.

15. A composition comprising the peptide of claim 11 and an active agent selected from the group consisting of a peptide, a protein, a nucleic acid, a double-stranded RNA, a hematopoietic, an antiinfective; an antidementia; an antiviral, an antitumoral, an antipyretic, an analgesic, an anti-inflammatory, an antiulcerative, an antiallergenic, an antidepressant, a psychotropic, a cardiotonic, an antiarrythmic, a vasodilator, an antihypertensive, a hypotensive diuretic, an antidiabetic, an anticoagulant, a cholesterol-lowering agent, a therapeutic for osteoporosis, a hormone, an antibiotic, a vaccine, a cytokine, a hormone, a growth factor, a cardiovascular factor, a cell adhesion factor, a central or peripheral nervous system factor, a humoral electrolyte factor, a hemal organic substance, a bone growth factor, a gastrointestinal factor, a kidney factor, a connective tissue factor, a sense organ factor, an immune system factor, a respiratory system factor, a genital organ factor, an androgen, an estrogen, a prostaglandin, a somatotropin, a gonadotropin, an interleukin, a steroid, a bacterial toxoid, hirugen, hirulos, hirudine, a monoclonal antibody, a polyclonal antibody, a humanized antibody, an antibody fragment, an immunoglobin, morphine, hydromorphone, oxymorphone, lovorphanol, levallorphan, codeine, nalmefene, nalorphine, nalozone, naltrexone, buprenorphine, butorphanol, or nalbufine, cortisone, hydrocortisone, fludrocortisone, prednisone, prednisolone, methylprednisolone, triamcinolone, dexamethoasone, betamethoasone, paramethosone, fluocinolone, colchicine, acetaminophen, a non-steroidal anti-inflammatory agent NSAID, acyclovir, ribavarin, trifluorothyridine, Ara-A Arabinofuranosyladenine, acylguanosine, nordeoxyguanosine, azidothymidine, dideoxyadenosine, dideoxycytidine, spironolactone, testosterone, estradiol, progestin, gonadotrophin, estrogen, progesterone, papaverine, nitroglycerin, a vasoactive intestinal peptide, calcitonin gene-related peptide, cyproheptadine, doxepin, imipramine, cimetidine, dextromethorphan, clozaril, superoxide dismutase, neuroenkephalinase, amphotericin B, griseofulvin, miconazole, ketoconazole, tioconazol, itraconazole, fluconazole, cephalosporin, tetracycline, aminoglucoside, erythromicin, gentamicin, polymyxin B, 5-fluorouracil, bleomycin, methotrexate, hydroxyurea, dideoxyinosine, floxuridine, 6-mercaptopurine, doxorubicin, daunorubicin, 1-darubicin, taxol, paclitaxel, tocopherol, quinidine, prazosin, verapamil, nifedipine, diltiazem, tissue plasminogen activator TPA, epidermal growth factor EGF, fibroblast growth factor FGF-acidic or basic, platelet derived growth factor PDGF, transforming growth factor TGF-alpha or beta, vasoactive intestinal peptide, tumor necrosis factor TNF, hypothalmic releasing factor, prolactin, thyroid stimulating hormone TSH, adrenocorticotropic hormone ACTH, parathyroid hormone PTH, follicle stimulating hormone FSF, luteinizing hormone releasing hormone LHRH, endorphin, glucagon, calcitonin, oxytocin, carbetocin, aldoetecone, enkaphalin, somatostin, somatotropin, somatomedin, alpha-melanocyte stimulating hormone, lidocaine, sufentainil, terbutaline, droperidol, scopolamine, gonadorelin, ciclopirox, buspirone, cromolyn sodium, midazolam, cyclosporin, lisinopril, captopril, delapril, ranitidine, famotidine, superoxide dismutase, asparaginase, arginase, arginine deaminease, adenosine deaminase ribonuclease, trypsin, chemotrypsin, papain, bombesin, substance P, vasopressin, alpha-globulins, transferrin, fibrinogen, beta-lipoprotein, beta-globulin, prothrombin, ceruloplasmin, alpha2-glycoprotein, alpha2-globulin, fetuin, alpha1-lipoprotein, alpha1-globulin, albumin, and prealbumin.

16. An isolated peptide comprising a sequence selected from the group consisting of
SEQ ID NOs:1 and 20-23, and variants thereof, having 14-26 amino acid residues, wherein the peptide is enriched with at least 60% lysine, leucine, and/or alanine, and pharmaceutically-acceptable salts thereof.

17. The peptide of claim 16, wherein the peptide has at least two D-amino acid residues.

18. The peptide of claim 16, the peptide comprising a sequence selected from the group consisting of SEQ ID NOs:3 and 17.

19. The peptide of claim 16, wherein the peptide is covalently linked to a polyethyleneglycol chain having a molecular size less than 40 kDa.

20. A composition comprising the peptide of claim 16 and an active agent selected from the group consisting of GLP-1, PYY3-36, PTH1-34, Exendin-4, beta-interferon, alpha-interferon, insulin, erythropoietin, G-CSF, GM-CSF, human growth hormone, and analogs thereof.

21. A composition comprising the peptide of claim 16 and an active agent selected from the group consisting of a peptide, a protein, a nucleic acid, a double-stranded RNA, a hematopoietic, an antiinfective; an antidementia; an antiviral, an antitumoral, an antipyretic, an analgesic, an anti-inflammatory, an antiulcerative, an antiallergenic, an antidepressant, a psychotropic, a cardiotonic, an antiarrythmic, a vasodilator, an antihypertensive, a hypotensive diuretic, an antidiabetic, an anticoagulant, a cholesterol-lowering agent, a therapeutic for osteoporosis, a hormone, an antibiotic, a vaccine, a cytokine, a hormone, a growth factor, a cardiovascular factor, a cell adhesion factor, a central or peripheral nervous system factor, a humoral electrolyte factor, a hemal organic substance, a bone growth factor, a gastrointestinal factor, a kidney factor, a connective tissue factor, a sense organ factor, an immune system factor, a respiratory system factor, a genital organ factor, an androgen, an estrogen, a prostaglandin, a somatotropin, a gonadotropin, an interleukin, a steroid, a bacterial toxoid, hirugen, hirulos, hirudine, a monoclonal antibody, a polyclonal antibody, a humanized antibody, an antibody fragment, an immunoglobin, morphine, hydromorphone, oxymorphone, lovorphanol, levallorphan, codeine, nalmefene, nalorphine, nalozone, naltrexone, buprenorphine, butorphanol, or nalbufine, cortisone, hydrocortisone, fludrocortisone, prednisone, prednisolone, methylprednisolone, triamcinolone, dexamethoasone, betamethoasone, paramethosone, fluocinolone, colchicine, acetaminophen, a non-steroidal anti-inflammatory agent NSAID, acyclovir, ribavarin, trifluorothyridine, Ara-A Arabinofuranosyladenine, acylguanosine, nordeoxyguanosine, azidothymidine, dideoxyadenosine, dideoxycytidine, spironolactone, testosterone, estradiol, progestin, gonadotrophin, estrogen, progesterone, papaverine, nitroglycerin, a vasoactive intestinal peptide, calcitonin gene-related peptide, cyproheptadine, doxepin, imipramine, cimetidine, dextromethorphan, clozaril, superoxide dismutase, neuroenkephalinase, amphotericin B, griseofulvin, miconazole, ketoconazole, tioconazol, itraconazole, fluconazole, cephalosporin, tetracycline, aminoglucoside, erythromicin, gentamicin, polymyxin B, 5-fluorouracil, bleomycin, methotrexate, hydroxyurea, dideoxyinosine, floxuridine, 6-mercaptopurine, doxorubicin, daunorubicin, 1-darubicin, taxol, paclitaxel, tocopherol, quinidine, prazosin, verapamil, nifedipine, diltiazem, tissue plasminogen activator TPA, epidermal growth factor EGF, fibroblast growth factor FGF-acidic or basic, platelet derived growth factor PDGF, transforming growth factor TGF-alpha or beta, vasoactive intestinal peptide, tumor necrosis factor TNF, hypothalmic releasing factor, prolactin, thyroid stimulating hormone TSH, adrenocorticotropic hormone ACTH, parathyroid hormone PTH, follicle stimulating hormone FSF, luteinizing hormone releasing hormone LHRH, endorphin, glucagon, calcitonin, oxytocin, carbetocin, aldoetecone, enkaphalin, somatostin, somatotropin, somatomedin, alpha-melanocyte stimulating hormone, lidocaine, sufentainil, terbutaline, droperidol, scopolamine, gonadorelin, ciclopirox, buspirone, cromolyn sodium, midazolam, cyclosporin, lisinopril, captopril, delapril, ranitidine, famotidine, superoxide dismutase, asparaginase, arginase, arginine deaminease, adenosine deaminase ribonuclease, trypsin, chemotrypsin, papain, bombesin, substance P, vasopressin, alpha-globulins, transferrin, fibrinogen, beta-lipoprotein, beta-globulin, prothrombin, ceruloplasmin, alpha2-glycoprotein, alpha2-globulin, fetuin, alpha1-lipoprotein, alpha1-globulin, albumin, and prealbumin.


SEQUENCE LISTING (24 sequences)

SEQ ID NO: 1 - protein, 18 aa, Artificial Sequence
  Description of Artificial Sequence Synthetic peptide
KLALKLALKALKAALKLA

SEQ ID NO: 2 - protein, 18 aa, Artificial Sequence
  Description of Artificial Sequence Synthetic peptide
KLALKLALKALKAALKLA

SEQ ID NO: 3 - protein, 15 aa, Artificial Sequence
  Description of Artificial Sequence Synthetic Peptide
LKLLKKLLKKLLKLL

SEQ ID NO: 4 - protein, 18 aa, Artificial Sequence
  Description of Artificial Sequence Synthetic peptide
KLALKLALKALKAALKLA

SEQ ID NO: 5 - protein, 18 aa, Artificial Sequence
  Description of Artificial Sequence Synthetic peptide
ALKLAAKLAKLALKLALK

SEQ ID NO: 6 - protein, 14 aa, Artificial Sequence
  Description of Artificial Sequence Synthetic peptide
KLALKLALKALKAA

SEQ ID NO: 7 - protein, 16 aa, Artificial Sequence
  Description of Artificial Sequence Synthetic peptide
KLALKLALKALKAALK

SEQ ID NO: 8 - protein, 20 aa, Artificial Sequence
  Description of Artificial Sequence Synthetic peptide
KLALKLALKALKAALKLALK

SEQ ID NO: 9 - protein, 22 aa, Artificial Sequence
  Description of Artificial Sequence Synthetic peptide
KLALKLALKALKAALKLALKLA

SEQ ID NO: 10 - protein, 24 aa, Artificial Sequence
  Description of Artificial Sequence Synthetic peptide
KLALKLALKALKAALKLALKLALK

SEQ ID NO: 11 - protein, 26 aa, Artificial Sequence
  Description of Artificial Sequence Synthetic peptide
KLALKLALKALKAALKLALKLALKAL

SEQ ID NO: 12 - protein, 18 aa, Artificial Sequence
  Description of Artificial Sequence Synthetic peptide
RLALRLALRALRAALRLA

SEQ ID NO: 13 - protein, 18 aa, Artificial Sequence
  Description of Artificial Sequence Synthetic peptide
RLAWRLALRALRAALRLA

SEQ ID NO: 14 - protein, 18 aa, Artificial Sequence
  Description of Artificial Sequence Synthetic peptide
KLAWKLALKALKAALKLA

SEQ ID NO: 15 - protein, 18 aa, Artificial Sequence
  Description of Artificial Sequence Synthetic peptide
KLAWKLALKALKAAWKLA

SEQ ID NO: 16 - protein, 18 aa, Artificial Sequence
  Description of Artificial Sequence Synthetic peptide
KLAWKLAWKALKAAWKLA

SEQ ID NO: 17 - protein, 15 aa, Artificial Sequence
  Description of Artificial Sequence Synthetic peptide
LKLLKKLLKKLLKLL

SEQ ID NO: 18 - protein, 15 aa, Artificial Sequence
  Description of Artificial Sequence Synthetic peptide
LKLLKKLLKKLLKLL

SEQ ID NO: 19 - protein, 18 aa, Artificial Sequence
  Description of Artificial Sequence Synthetic peptide
LKTLATALTKLAKTLTTL

SEQ ID NO: 20 - protein, 18 aa, Artificial Sequence
  Description of Artificial Sequence Synthetic peptide
KALKLKAALALLAKLKLA

SEQ ID NO: 21 - protein, 18 aa, Artificial Sequence
  Description of Artificial Sequence Synthetic peptide
KLAAALLKKAKKLAAALL

SEQ ID NO: 22 - protein, 18 aa, Artificial Sequence
  Description of Artificial Sequence Synthetic peptide
KALAALLKKAAKLLAALK

SEQ ID NO: 23 - protein, 18 aa, Artificial Sequence
  Description of Artificial Sequence Synthetic peptide
KALAALLKKLAKLLAALK

SEQ ID NO: 24 - protein, 18 aa, Artificial Sequence
  Description of Artificial Sequence Synthetic peptide
KLALKLALKNLKAALKLA